(19) United States
(12) Patent Application Publication
(10) Pub. No.: US 2006/0167075 A1
(43) Pub. Date: Jul. 27, 2006

(54) Modulators of FAAH

(76) Inventors: James Philip Pearson, Cambridge, MA (US); G. Todd Milne, Brookline, MA (US); Mark G. Currie, Sterling, MA (US); Brian M. Cali, Arlington, MA (US); John Jeffrey Talley, Somerville, MA (US)

Correspondence Address: FISH & RICHARDSON PC, P.O. BOX 1022, MINNEAPOLIS, MN, 55440-1022, US

(21) Appl. No.: 11/340,232

(22) Filed: Jan. 25, 2006

Related U.S. Application Data
(60) Provisional application No. 60/647,165, filed on Jan. 25, 2005.

(51) Int. Cl. A61K 31/415; A61K 31/365

(52) U.S. Cl. 514/406; 514/471

(57) ABSTRACT

Methods for inhibiting the activity of FAAH and methods for preventing and/or treating certain disorders, e.g., anxiety disorders, sleep disorders and weight disorders, by administering celecoxib, valdecoxib or certain structurally related compounds at a dosage that is sufficient to treat and/or prevent the disorder are described.

### CLAIM OF PRIORITY

This application claims priority under 35 USC §119(e) to U.S. Provisional Patent Application Ser. No. 60/647,165, filed on Jan. 25, 2005, the entire contents of which is hereby incorporated by reference.

### BACKGROUND

FAAH (fatty acid amide hydrolase) is a serine hydrolase that has been shown to be important for the hydrolysis of the fatty acid amide (FAA) class of endogenous, lipid signaling molecules. FAAH has been shown to be relevant to the in vivo degradation of anandamide (AEA), oleamide, N-palmitoyl ethanolamide (PEA), and N-oleoyl ethanolamide (OEA). These molecules act through a number of pathways and control diverse physiological behaviors including anxiety, pain, satiety, and sleep.

The biological consequences of FAAH inhibition have been addressed both genetically through a knockout mouse and by small molecule inhibitors of the enzyme. The FAAH ±KO (knockout) mouse clearly demonstrates the importance of FAAH in the turnover of AEA, PEA, oleamide, and OEA as FAAH −/− mice degrade these FAAs 5-100× less rapidly than wild type littermates (Cravatt et al, Chemistry and Physics of Lipids, 2002, 121:135-148). Loss of FAAH activity had the biological consequence of potentiating the cannabimimetic effects of exogenously introduced AEA—antinociception, hypothermia, and hypomotility. These effects are mediated through the CB1 receptor as they are blocked by the administration of SR141716A, a potent CB1 antagonist. However, in the absence of exogenous AEA, Cravatt et al noted that the knockout mice were “viable, fertile, and largely indistinguishable from littermates” with no differences observed in “general appearance, body weight, locomotion, or overt behavior” (Cravatt, et al, Proc. Natl. Acad Sci., 2001, 98(16):9371-6). The brains of FAAH −/− mice do contain elevated levels of endogenous AEA, PEA, and OEA and the mice themselves are less responsive to painful stimuli. These FAAH −/− mice also possess higher slow-wave sleep values and exhibit more intense episodes of slow-wave sleep than control littermates (Huitron-Resendiz, et al, Sleep, 2004, 27(5):857-865). In addition, in the absence of FAAH activity (in the FAAH −/− mouse), anandamide is converted to prostamides in what is likely a COX-dependent mechanism (Weber, A, et al, 2004, J Lipid Res, 45:757-763). The pharmacology of prostamides suggests that increasing prostamide levels may be beneficial for the treatment of diseases of the eye characterized by ocular hypertension such as glaucoma.

A number of small molecule inhibitors of FAAH have been identified and tested in animal models. These data clearly demonstrate that FAAH can be inhibited by small molecules and that inhibition of FAAH in vivo has profound anxiolytic effects. As in the FAAH KO mouse, treatment of rats with known inhibitors of FAAH such as PMSF, URB532, or URB597, potentiates the cannabimimetic effects of exogenously added AEA (Kathuria et al, Nature Medicine, 2003, 9(1):76-81). However, in the absence of exogenous FAA administration, the outward biological consequences of FAAH inhibition are relatively subtle—there are no signs of catalepsy, hypothermia, or hyperphagia as would be expected from administration of a CB1 agonist. Treatment with URB532 or URB597 does result in significant elevations in endogenous brain levels of AEA, OEA, and PEA (Kathuria et al, Nature Medicine, 2003, 9(1):76-81). There is also a moderate decrease in pain sensitivity (hot plate test). Importantly, URB532 or URB597 treated mice show dramatic CB1-dependent decreases in anxiety in both the elevated zero maze and isolation-induced ultrasonic emission test. Changes in sleep patterns and satiety cues might also be expected based on elevated levels of fatty acid amides relative to untreated animals.

### SUMMARY

The invention features methods for inhibiting the activity of FAAH and methods for preventing and/or treating certain disorders, e.g., anxiety disorders, sleep disorders and weight disorders, by administering celecoxib, valdecoxib or certain structurally related compounds at a dosage that is sufficient to treat and/or prevent the disorder. Celecoxib and valdecoxib are inhibitors of COX-2 and are commonly used to treat inflammation. However, the present inventors have found that at higher concentrations these compounds inhibit FAAH and are thus useful in the prevention and/or treatment of pain, anxiety and certain other disorders. The compounds may also be useful for treating anxiety disorders, sleep disorders and weight disorders when administered at dosage at which FAAH is not detectably inhibited.

The invention also features a method comprising administering celecoxib, valdecoxib, a compound structurally related to celecoxib or valdecoxib or a pharmaceutical composition comprising celecoxib, valdecoxib or a compound structurally related to celecoxib or valdecoxib to achieve a serum concentration sufficient to prevent and/or treat a disorder associated with undesirable FAAH activity.

In another aspect the invention features a method for preventing and/or treating anxiety comprising administering celecoxib, valdecoxib, a compound structurally related to celecoxib or valdecoxib or a pharmaceutical composition comprising celecoxib, valdecoxib or a compound structurally related to celecoxib or valdecoxib to achieve a serum concentration sufficient to prevent and/or treat a sleep disorder, e.g., insomnia. In certain embodiments, the method comprises administering celecoxib, valdecoxib, a compound structurally related to celecoxib or valdecoxib or a pharmaceutical composition comprising celecoxib, valdecoxib or a compound structurally related to celecoxib or valdecoxib to achieve a serum concentration sufficient to inhibit FAAH activity.

In another aspect the invention features a method for preventing and/or treating a sleep disorder comprising administering celecoxib, valdecoxib, a compound structurally related to celecoxib or valdecoxib or a pharmaceutical composition comprising celecoxib, valdecoxib or a compound structurally related to celecoxib or valdecoxib to achieve a serum concentration sufficient to prevent and/or treat a sleep disorder, e.g., insomnia. In certain embodiments, the method comprises administering celecoxib, valdecoxib, a compound structurally related to celecoxib or valdecoxib or a pharmaceutical composition comprising celecoxib, valdecoxib or a compound structurally related to celecoxib or valdecoxib to achieve a serum concentration sufficient to inhibit FAAH activity.

In another aspect the invention features a method for reducing FAAH activity comprising administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to inhibit FAAH activity.

Inhibition of FAAH activity can be measured, for example, by measuring elevation in levels of fatty acid amides such as anandamide (AEA), oleamide, N-palmitoyl ethanolamide (PEA), and N-oleoyl ethanolamide (OEA).

In another aspect the invention features a method for treating glaucoma comprising administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to prevent and/or treat glaucoma. In certain embodiments, the method comprises administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to inhibit FAAH activity.

In another aspect the invention features a method for treating intraocular hypertension comprising administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to prevent and/or treat intraocular hypertension. In certain embodiments, the method comprises administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to inhibit FAAH activity.

In another aspect the invention features a method for treating a weight disorder comprising administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to and prevent and/or treat a weight disorder, e.g., obesity. In certain embodiments, the method comprises administering celecoxib, valdecoxib, a compound that is structurally related to celecoxib or valdecoxib or a pharmaceutical composition comprising celecoxib, valdecoxib or a compound that is structurally related to celecoxib or valdecoxib to achieve a serum concentration sufficient to inhibit FAAH activity.

For inhibition of FAAH activity, valdecoxib can be administered at a dosage of 25, 30, 40, 50, 60, 70, 80, 90 or 100 mg/day. For inhibition of FAAH activity, celecoxib can be administered at a dosage of 400, 450, 500, 550, 600, 700, 750, 800, 850, 900, 950, or 1,000 mg/day. In certain embodiments, the dosage is greater than that required to inhibit COX-2 activity.

The invention also features a method inhibiting FAAH in a patient, the method comprising administering celecoxib, valdecoxib a compound structurally related to celecoxib or valdecoxib or a pharmaceutical composition comprising celecoxib, valdecoxib a compound structurally related to celecoxib or valdecoxib to a patient at a dosage sufficient to inhibit FAAH activity in the patient.

In various embodiments of the methods of the invention FAAH activity is inhibited at least 10%, 20%, 40%, 50%, 70% or more.

The invention also features a method for treating anxiety in a patient, comprising: (a) identifying a patient as suffering from anxiety or at risk of suffering from anxiety; and (b) administering celecoxib, valdecoxib, a compound structurally related to celecoxib or valdecoxib or a pharmaceutical composition comprising celecoxib, valdecoxib or a compound structurally related to celecoxib or valdecoxib to the patient.

In another aspect the invention features a method for treating a sleep disorder in a patient, the method comprising: (a) identifying a patient as suffering from pain or at risk of suffering from a sleep disorder; and (b) administering celecoxib, valdecoxib, a compound structurally related to celecoxib or valdecoxib or a pharmaceutical composition comprising celecoxib, valdecoxib or a compound structurally related to celecoxib or valdecoxib to the patient.

In another aspect the invention features a method for treating a weight disorder in a patient, the method comprising: (a) identifying a patient as suffering from a weight disorder; and (b) administering celecoxib, valdecoxib, a compound structurally related to celecoxib or valdecoxib or a pharmaceutical composition comprising celecoxib, valdecoxib or a compound structurally related to celecoxib or valdecoxib to the patient.

In another aspect the invention features a method for treating intraocular hypertension in a patient, the method comprising: (a) identifying a patient as suffering from intraocular hypotension or at risk of suffering from intraocular hypertension; and (b) administering celecoxib, valdecoxib, a compound structurally related to celecoxib or valdecoxib or a pharmaceutical composition comprising celecoxib, valdecoxib or a compound structurally related to celecoxib or valdecoxib to the patient.

In various embodiments of the methods the celecoxib, valdecoxib, a compound structurally related to celecoxib or valdecoxib or a pharmaceutical composition comprising celecoxib, valdecoxib or a compound structurally related to celecoxib or valdecoxib is administered at a dosage sufficient to inhibit FAAH activity.

In certain embodiments the compounds are administered in combination with a second compound, e.g., a compound useful for reducing anxiety, reducing pain, or treating a weight or sleep disorder.

The subject can be a mammal, preferably a human. Identifying a subject in need of such treatment can be in the judgment of a subject or a health care professional and can be subjective (e.g., opinion) or objective (e.g., measurable by a test or diagnostic method).

The term “mammal” includes, for example, mice, hamsters, rats, cows, sheep, pigs, goats, and horses, monkeys, dogs (e.g., Canis familiaris), cats, rabbits, guinea pigs, and primates, including humans.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.05 mg/Kg to about 500 mg/Kg, alternatively from about 1 to about 50 mg/Kg. Effective doses will also vary depending on route of administration, as well as the possibility of co-usage with other agents.

The invention includes the use of salts, particularly physiologically acceptable salts, and solvates of celecoxib and valdecoxib. Solvates are forms of the compounds in which the compound forms a complex with solvent molecules by coordination in the solid or liquid states. Hydrates are a special form of solvate in which the compound is coordinated with water. The invention also includes the use of enantiomers, diastereomers and mixtures thereof.

Additional compounds useful include certain compounds that are structurally related to celecoxib or valdecoxib.

Compounds having Formula I, below, can be useful in the methods described herein. 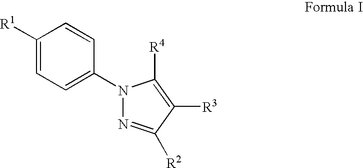
wherein: R¹ is sulfamyl; wherein R² is haloalkyl; wherein R³ is selected from hydrido, and alkyl; and wherein R⁴ is selected from aryl, cycloalkyl, and cycloalkenyl; wherein R⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy, hydroxyalkyl, haloalkoxy, sulfamyl, N-alkylsulfamyl, amino, N-alkylamino, N,N-dialkylamino, heterocyclic, nitro and acylamino; and pharmaceutically-acceptable salts thereof.

In other embodiments, R² is lower haloalkyl; R³ is hydrido; and wherein R⁴ is selected from aryl, cycloalkyl, and cycloalkenyl; wherein R⁴ is optionally substituted at a substitutable position with one or more radicals selected from halo, lower alkylthio, lower alkylsulfinyl, lower alkyl, lower alkylsulfonyl, cyano, carboxyl, lower alkoxycarbonyl, amido, lower N-monoalkylamido, N-monoarylamido, lower N,N-dialkylamido, lower N-alkyl-N-arylamido, lower haloalkyl, hydroxyl, lower alkoxy, lower hydroxyalkyl, lower haloalkoxy, sulfamyl, lower N-alkylsulfamyl, amino, lower N-alkylamino, lower N,N-dialkylamino, heterocyclic, nitro and acylamino; or a pharmaceutically-acceptable salt thereof.

In other embodiments, R² is selected from fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, and dichloropropyl; R³ is hydrido; and R⁴ is selected from phenyl, naphthyl, biphenyl, cyclohexyl, cyclopentyl, cycloheptyl, 1-cyclohexenyl, 2-cyclohexenyl, 3-cyclohexenyl, 4-cyclohexenyl, and 1-cyclopentenyl; wherein R⁴ is optionally substituted at a substitutable position with one or more radicals selected from fluoro, chloro, bromo, methylthio, methylsulfinyl, cyano, carboxyl, amido, methoxycarbonyl, ethoxycarbonyl, isopropoxycarbonyl, tertbutoxycarbonyl, propoxycarbonyl, butoxycarbonyl, isobutoxycarbonyl, pentoxycarbonyl, N-methylamido, N-ethylamido, N-isopropylamido, N-propylamido, N-butylamido, N-isobutylamido, N-tert-butylamido, N-pentylamido, N-cyclohexylamido, N-cyclopentylamido, N,N-dimethylamido, N-methyl-N-ethylamido, pyrrolidinoamido, piperidinoamido, N-phenylamido, N-(3-fluorophenyl)amido, N-(4-methylphenyl)amido, N-(3-chlorophenyl)amido, N-(4-methoxyphenyl)amido, 2-pyridylamido, N-methyl-N-phenylamido, N-methyl-N-pyridylamido, methyl, ethyl, isopropyl, tert-butyl, isobutyl, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl, hydroxyl, methoxy, methylenedioxy, ethoxy, propoxy, n-butoxy, trifluoromethoxy, hydroxymethyl, hydroxymethyl, hydroxypropyl, sulfamyl, methylsulfamyl, amino, nitro, methylamino, dimethylamino, formylamino, acetamino, trifluoroacetamino and morpholino; or a pharmaceutically-acceptable salt thereof.

In other embodiments, R¹ is sulfamyl; R² is lower haloalkyl; wherein R³ is lower alkyl; and R⁴ is selected from aryl, cycloalkyl, and cycloalkenyl; wherein R⁴ is optionally substituted at a substitutable position with one or more radicals selected from halo, lower alkylthio, lower alkylsulfinyl, lower alkyl, lower alkylsulfonyl, cyano, carboxyl, lower alkoxycarbonyl, amido, lower N-monoalkylamido, N-monoarylamido, lower N,N-dialkylamido, lower N-alkyl-N-arylamido, lower haloalkyl, hydroxyl, lower alkoxy, lower hydroxyalkyl, lower haloalkoxy, sulfamyl, lower N-alkylsulfamyl, amino, lower N-alkylamino, lower N,N-dialkylamino, heterocyclic, nitro and acylamino; or a pharmaceutically-acceptable salt thereof.

In other embodiments, R² is lower haloalkyl; R³ is lower alkyl; and R⁴ is aryl optionally substituted at a substitutable position with halo; or a pharmaceutically-acceptable salt thereof.

In other embodiments, R² is selected from fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl; R³ is selected from methyl, ethyl, propyl, isopropyl, and butyl; and R⁴ is phenyl optionally substituted at a substitutable position with one or more radicals selected from fluoro, chloro and bromo; or a pharmaceutically-acceptable salt thereof.

In other embodiments, R¹ is H₂NHSO₂—; R² is lower haloalkyl; R³ is hydrido; and R⁴ is selected from aryl, cycloalkyl, and cycloalkenyl; wherein R⁴ is optionally substituted at a substitutable position with one or more radicals selected from halo, lower alkylthio, lower alkylsulfonyl, cyano, nitro, lower haloalkyl, lower alkyl, hydrido, lower alkoxy, lower haloalkoxy, sulfamyl, heterocyclic and amino; or a pharmaceutically-acceptable salt thereof. In certain of these embodiments, wherein R² is selected from fluoromethyl, difluoromethyl, trifluoromethyl, chloromethyl, dichloromethyl, trichloromethyl, pentafluoroethyl, heptafluoropropyl, difluorochloromethyl dichlorofluoromethyl, difluoroethyl, difluoropropyl, dichloroethyl, dichloropropyl; R³ is hydrido; and R⁴ is selected from phenyl, biphenyl, cyclohexyl, and cyclohexenyl; wherein R⁴ is optionally substituted at a substitutable position with one or more radicals selected from chloro, bromo, fluoro, methylthio, methylsulfonyl, morpholinyl, amino, nitro, methyl, ethyl, propyl, isopropyl, butyl, methoxy, ethoxy, propoxy, butoxy, trifluoromethoxy, trifluoroethoxy, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl; or a pharmaceutically-acceptable salt thereof.

Useful compounds of Formula I can include: 4-[5-(4-methylphenyl)-3-(trifluoromethyl)-1H-pyrazol-1-yl]benzenesulfonamide; 4-[5-(3,4-dichlorophenyl)-3-(trifluoromethyl)-1H-pyrazol-1-yl]benzenesulfonamide; 4-[5-(4-chlorophenyl)-3-(trifluoromethyl)-1H-pyrazol-1-yl]benzenesulfonamide; 4-[5-(4-bromophenyl)-3-(trifluoromethyl)-1H-pyrazol-1-yl]benzenesulfonamide; 4-[5-(3-chlorophenyl)-3-(trifluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[5-(2-chlorophenyl)-3-(trifluoromethyl)-1H-pyrazol-1-yl]benzenesulfonamide; 4-[5-(2-fluorophenyl)-3-(trifluoromethyl)-1H-pyrazol-1-yl]benzenesulfonamide; 4-[5-[4-(trifluoromethyl)phenyl]-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-[4-(trifluoromethoxy)phenyl]-3-(trifluoromethyl)-1H-pyrazol-1-yl]benzenesulfonamide; 4-[5-(2-methylphenyl)-3-(trifluoromethyl)-1H-pyrazol-1-yl]benzenesulfonamide; 4-[5-(4-nitrophenyl)-3-(trifluoromethyl)-1H-pyrazol-1-yl]benzenesulfonamide; 4-[5-(4-aminophenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-[4(methylthio)phenyl]-3-(difluoromethyl)-1H-pyrazol-1-yl]benzenesulfonamide; 4-[5-(2,4-difluorophenyl)-3-(trifluoromethyl)-1H-pyrazol-1-yl]benzenesulfonamide; 4-[5-(2,6-difluorophenyl)-3-(trifluoromethyl)-1H-pyrazol-1-yl]benzenesulfonamide; 4-[5-(4-chlorophenyl)-3-(heptafluoropropyl)-1H-pyrazol-1-yl]benzenesulfonamide; 4-[5-(4-chlorophenyl)-3-(chloro-difluoromethyl)-1H-pyrazol-1-yl]benzenesulfonamide; 4-[5-(4-chlorophenyl)-3-(pentafluoroethyl)-1H-pyrazol-1-yl]benzenesulfonamide; 4-[5-(4-biphenyl)-3-(difluoromethyl)-1H-pyrazol-1-yl]benzenesulfonamide; 4-[5-[4-(morpholino)phenyl]-3-(difluoromethyl)-1H-pyrazol-1-yl]benzenesulfonamide; 4-[5-(1-cyclohexenyl)-3-(difluoromethyl)-1H-pyrazol-1-yl]benzenesulfonamide; 4-[5-(1-cyclohexyl)-3-(difluoromethyl)-1H-pyrazol-1-yl]benzenesulfonamide; 4-[5-[4-(trifluoromethyl)phenyl] -3-(difluoromethyl)-1H-pyrazol-1-yl]benzenesulfonamide; 4-[5-(3,4-dichlorophenyl)-3-(trifluoromethyl)-1H-pyrazol-1-yl]benzenesulfonamide; 4-[5-(2,4-dichlorophenyl)-3-(trifluoromethyl)-1H-pyrazol-1-yl]benzenesulfonamide; 4-[5-(4-chlorophenyl)-3-(fluoromethyl)-1H-pyrazol-1-yl]benzenesulfonamide; 4-[5-(4-chlorophenyl)-3-(chloromethyl)-1H-pyrazol-1-yl]benzenesulfonamide; 4-[5-(4-chlorophenyl)-3-(dichloromethyl)-1H-pyrazol-1-yl]benzenesulfonamide; 4-[5-(4-chlorophenyl)-3-(dichlorofluoromethyl)-1H-pyrazol-1-yl]benzenesulfonamide; 4-[5-(3,5-difluorophenyl)-3-(trifluoromethyl)-1H-pyrazol-1-yl]benzenesulfonamide; 4-[5-(2,4,6-trifluorophenyl)-3-(trifluoromethyl)-1H-pyrazol-1-yl]benzenesulfonamide; 4-[5-(2,6-dichlorophenyl)-3-(trifluoromethyl)-1H-pyrazol-1-yl]benzenesulfonamide; 4-[5-(2,4,6-trichlorophenyl)-3-(trifluoromethyl)-1H-pyrazol-1-yl]benzenesulfonamide; 4-[5-(2-methoxyphenyl)-3-(trifluoromethyl)-1H-pyrazol-1-yl]benzenesulfonamide; 4-[5-(3-methoxyphenyl)-3-(trifluoromethyl)-1H-pyrazol-1-yl]benzenesulfonamide; 4-[5-(3,4-dimethoxyphenyl)-3-(trifluoromethyl)-1H-pyrazol-1-yl]benzenesulfonamide; 4-[5-(3,4-methylenedioxyphenyl)-3-trifluoromethyl)-1H-pyrazol-1-yl]benzenesulfonamide; 4-[5-(2-fluoro-4-methoxyphenyl)-3-trifluoromethyl)-1H-pyrazol-1-yl]benzenesulfonamide; 4-[5-(3-fluoro-4-methoxyphenyl)-3-trifluoromethyl)-1H-pyrazol-1-yl]benzenesulfonamide; 4-[5-(4-fluoro-2-methoxyphenyl)-3-trifluoromethyl)-1H-pyrazol-1-yl]benzenesulfonamide; 4-[5-(2-chloro-4-methoxyphenyl)-3-trifluoromethyl)-1H-pyrazol-1-yl]benzenesulfonamide; 4-[5-(4-chloro-2-methoxyphenyl)-3-trifluoromethyl)-1H-pyrazol-1-yl]benzenesulfonamide; 4-[5-(2-methythiophenyl)-3-(trifluoromethyl)-1H-pyrazol-1-yl]benzenesulfonamide; 4-[5-(3-methythiophenyl)-3-(trifluoromethyl)-1H-pyrazol-1-yl]benzenesulfonamide; 4-[5-(4-methythiophenyl)-3-(trifluoromethyl)-1H-pyrazol-1-yl]benzenesulfonamide; 4-[5-(2-methylsulfinylphenyl)-3-(trifluoromethyl)-1H-pyrazol-1-yl]benzenesulfonamide; 4-[5-(3-methylsulfinylphenyl)-3-(trifluoromethyl)-1H-pyrazol-1-yl]benzenesulfonamide; 4-[5-(4-methylsulfinylphenyl)-3-(trifluoromethyl)-1H-pyrazol-1-yl]benzenesulfonamide; 4-[5-(3-methylphenyl)-3-(trifluoromethyl)-1H-pyrazol-1-yl]benzenesulfonamide; 4-[5-(2-fluoro-4-methylphenyl)-3-(trifluoromethyl)-1H-pyrazol-1-yl]benzenesulfonamide; 4-[5-(4-fluoro-3-methylphenyl)-3-(trifluoromethyl)-1H-pyrazol-1-yl]benzenesulfonamide; 4-[5-(2-chloro-4-methylphenyl)-3-(trifluoromethyl)-1H-pyrazol-1-yl]benzenesulfonamide; 4-[5-(4-chloro-2-methylphenyl)-3-(trifluoromethyl)-1H-pyrazol-1-yl]benzenesulfonamide; 4-[5-(4-hydroxyphenyl)-3-(trifluoromethyl)-1H-pyrazol-1-yl]benzenesulfonamide; 4-[5-(3,4-dihydroxyphenyl)-3-(trifluoromethyl)-1H-pyrazol-1-yl]benzenesulfonamide; 4-[5-biphenyl-3-(trifluoromethyl)-1H-pyrazol-1-yl]benzenesulfonamide; 4-[5-(4-ethylphenyl)-3-(trifluoromethyl)-1H-pyrazol-1-yl]benzenesulfonamide; 4-[5-(4-isopropylphenyl)-3-(trifluoromethyl)-1H-pyrazol-1-yl]benzenesulfonamide; 4-[5-(6-methoxy-2-naphthyl)-3-(trifluoromethyl)-1H-pyrazol-1-yl]benzenesulfonamide; 4-[5-(2-naphthyl)-3-(trifluoromethyl)-1H-pyrazol-1-yl]benzenesulfonamide; 4-[5-[4-(N-ethylamino)phenyl)]-3-trifluoromethyl)-1H-pyrazol-1-yl]benzenesulfonamide; 4-[5-[4-(N,N-dimethylamino)phenyl]-3-(trifluoromethyl)-1H-pyrazol-1-yl]benzenesulfonamide; 4-[5-[4-(N-formylamino)phenyl]-3-(trifluoromethyl)-1H-pyrazol-1-yl]benzenesulfonamide; 4-[5-[4-(N-acetamino)phenyl]-3-(trifluoromethyl)-1H-pyrazol-1-yl]benzenesulfonamide; 4-[5-[4-(N-methylsulfonamido)phenyl]-3-(trifluoromethyl)-1H-pyrazol-1-yl]benzenesulfonamide; 4-[5-(4-hydroxymethylphenyl)-3-(trifluoromethyl)-1H-pyrazol-1-yl]benzenesulfonamide; 4-[5-(cyclohexyl)-3-(trifluoromethyl)-1H-pyrazol-1-yl]benzenesulfonamide; 4-[5-(cyclopentyl)-3-(trifluoromethyl)-1H-pyrazol-1-yl]benzenesulfonamide; 4-[5-(cycloheptyl)-3-(trifluoromethyl)-1H-pyrazol-1-yl]benzenesulfonamide; 4-[5-(1-cyclohexenyl)-3-(trifluoroethyl)-1H-pyrazol-1-yl]benzenesulfonamide; 4-[5-(1-cyclopentenyl)-3-(trifluoromethyl)-1H-pyrazol-1-yl]benzenesulfonamide; 4-[5-(4-chlorophenyl)-3-(trifluoromethyl)-4-(methyl)-1H-pyrazol-1-yl]benzenesulfonamide; 4-[5-(4-fluorophenyl)-3-(trifluoromethyl)-4-(n-propyl)-1H-pyrazol-1-yl]benzenesulfonamide; 4-[5-(4-fluorophenyl)-3-(trifluoromethyl)-4-(methyl)-1H-pyrazol-1-yl]benzensulfonamide; 4-[5-(4-chlorophenyl)-3-(trifluoromethyl)-1H-pyrazol-1-yl]benzenesulfonamide; 4-[5-(4-bromophenyl)-3-(trifluoromethyl)-1H-pyrazol-1-yl]benzenesulfonamide; 4-[5-(3-chlorophenyl)-3-(trifluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[5-(2-chlorophenyl)-3-(trifluoromethyl)-1H-pyrazol-1-yl]benzenesulfonamide; 4-[5-(2-fluorophenyl)-3-(trifluoromethyl)-1H-pyrazol-1-yl]benzenesulfonamide; 4-[5-[4-(trifluoromethyl)phenyl]-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-[4-(trifluoromethoxy)phenyl]-3-(trifluoromethyl)-1H-pyrazol-1-yl]benzenesulfonamide; 4-[5-(2-methylphenyl)-3-(trifluoroethyl)-1H-pyrazol-1-yl]benzenesulfonamide; 4-[5-(4-nitrophenyl)-3-(trifluoromethyl)-1H-pyrazol-1-yl]benzenesulfonamide; 4-[5-(4-aminophenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-(4-methylthiophenyl)-3-(difluoromethyl)-1H-pyrazol-1-yl]benzenesulfonamide; 4-[5-(2,4-difluorophenyl)-3-(trifluoromethyl)-1H-pyrazol-1-yl]benzenesulfonamide; 4-[5-(2,6-difluorophenyl)-3-(trifluoromethyl)-1H-pyrazol-1-yl]benzenesulfonamide; 4-[5-(4-chlorophenyl)-3-(heptafluoropropyl)-1H-pyrazol-1-yl]benzenesulfonamide; 4-[5-(4-chlorophenyl)-3-(chloro-difluoromethyl)-1H-pyrazol-1-yl]benzenesulfonamide; 4-[5-(4-chlorophenyl)-3-(pentafluoroethyl)-1H-pyrazol-1-yl]benzenesulfonamide; 4-[5-(4-biphenyl)-3-(difluoromethyl)-1H-pyrazol-1-yl]benzenesulfonamide; 4-[5-(4-(morpholino)phenyl)-3-(difluoromethyl)-1H-pyrazol-1-yl]benzenesulfonamide; 4-[5-(1-cyclohexenyl)-3-(difluoromethyl)-1H-pyrazol-1-yl]benzenesulfonamide; 4-[5-(1-cyclohexyl)-3-(difluoromethyl)-1H-pyrazol-1-yl]benzenesulfonamide; 4-[5-(4-[trifluoromethyl]phenyl)-3-(difluoromethyl)-1H-pyrazol-1-yl]benzenesulfonamide; 4-[5-(3,4-dichlorophenyl)-3-(trifluoromethyl)-1H-pyrazol-1-yl]benzenesulfonamide; 4-[5-(2,4-dichlorophenyl)-3-(trifluoromethyl)-1H-pyrazol-1-yl]benzenesulfonamide; and pharmaceutically acceptable salts thereof.

Compounds having Formula II or Formula IIA, below, can also be useful. 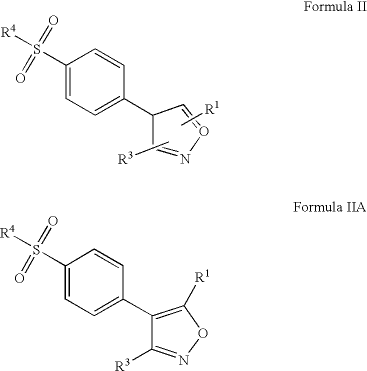
wherein: R¹ is selected from alkyl, carboxyalkyl, alkoxycarbonyl, aminocarbonyl, aminocarbonylalkyl, alkoxycarbonylalkyl, carboxyl, alkoxy, haloalkoxy, aralkoxy, cycloalkylalkoxy, alkylthio, aralkylthio, cycloalkylalkylthio, alkoxyalkyl, aralkoxyalkyl, alkylthioalkyl, aralkylthioalkyl, alkylaminoalkyl, aryloxyalkyl, arylthioalkyl, hydroxyl, amino, hydroxyalkyl, haloalkyl, cycloalkyl, cycloalkylalkyl, aralkyl, halo, alkylamino, aralkylamino, N-alkyl-N-aralkylamino, N-alkyl-N-cycloalkylalkylamino, arylcarbonyloxyalkyl, arylcarbonylthio, alkoxycarbonyloxyalkyl, alkylaminocarbonyloxyalkyl, alkoxycarbonylthioalkyl, and alkylaminocarbonylthioalkyl; R³ is selected from cycloalkyl, cycloalkenyl, and aryl, wherein R³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and R⁴ is selected from lower alkyl, hydroxyl, and amino; and pharmaceutically-acceptable salts thereof.

In certain embodiments, R⁴ is NH₂—.

In certain embodiments, R¹ is selected from alkyl, carboxyalkyl, alkoxycarbonyl, aminocarbonyl, aminocarbonylalkyl, alkoxycarbonylalkyl, carboxyl, alkoxy, haloalkoxy, aralkoxy, cycloalkylalkoxy, alkylthio, aralkylthio, cycloalkylalkylthio, alkoxyalkyl, aralkoxyalkyl, alkylthioalkyl, aralkylthioalkyl, alkylaminoalkyl, aryloxyalkyl, arylthioalkyl, hydroxyl, amino, hydroxyalkyl, haloalkyl, cycloalkyl, cycloalkylalkyl, aralkyl, halo, alkylamino, aralkylamino, N-alkyl-N-aralkylamino, N-alkyl-N-cycloalkylalkylamino, arylcarbonyloxyalkyl, arylcarbonylthio, alkoxycarbonyloxyalkyl, alkylaminocarbonyloxyalkyl, alkoxycarbonylthioalkyl, and alkylaminocarbonylthioalkyl; R³ is selected from cycloalkyl, cycloalkenyl, and aryl; wherein R³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and R⁴ is selected from lower alkyl, hydroxyl, and amino; or a pharmaceutically-acceptable salt thereof.

In other embodiments, R¹ is selected from hydroxyl, amino, lower alkyl, lower carboxyalkyl, lower alkoxycarbonyl, aminocarbonyl, carboxyl, lower aminocarbonylalkyl, lower alkoxycarbonylalkyl, lower alkoxy, lower haloalkoxy, lower aralkoxy, lower cycloalkylalkoxy, lower alkylthio, lower aralkylthio, lower cycloalkylalkylthio, lower alkoxyalkyl, lower aralkoxyalkyl, lower alkylthioalkyl, lower aralkylthioalkyl, lower alkylaminoalkyl, lower aryloxyalkyl, lower arylthioalkyl, lower hydroxyalkyl, lower haloalkyl, lower cycloalkyl, lower cycloalkylalkyl, lower aralkyl, halo, lower alkylamino, lower aralkylamino, lower N-alkyl-N-aralkylamino, lower N-alkyl-N-cycloalkylalkylamino, lower arylcarbonyloxyalkyl, lower alkoxycarbonyloxyalkyl, lower alkylaminocarbonyloxyalkyl, lower alkoxycarbonylthioalkyl, and lower alkylaminocarbonylthioalkyl; R³ is selected from cycloalkyl, cycloalkenyl, and aryl; wherein R³ is optionally substituted at a substitutable position with one or more radicals independently selected from lower alkylsulfinyl, lower alkyl, cyano, carboxyl, lower alkoxycarbonyl, lower haloalkyl, hydroxyl, lower hydroxyalkyl, lower haloalkoxy, amino, lower alkylamino, lower arylamino, lower aminoalkyl, nitro, halo, lower alkoxy, lower alkylsulfonyl, aminosulfonyl, and lower alkylthio; and R⁴ is selected from methyl, hydroxyl and amino; or a pharmaceutically-acceptable salt thereof.

In other embodiments, R¹ is selected from hydroxyl, lower alkyl, carboxyl, lower carboxyalkyl, lower aminocarbonylalkyl, lower alkoxycarbonylalkyl, lower aralkyl, lower alkoxyalkyl, lower aralkoxyalkyl, lower alkylthioalkyl, lower aralkylthioalkyl, lower alkylaminoalkyl, lower aryloxyalkyl, lower arylthioalkyl, lower haloalkyl, lower hydroxylalkyl, cycloalkyl, cycloalkylalkyl, and aralkyl; R³ is selected from cycloalkyl, cycloalkenyl, and aryl; wherein R³ is optionally substituted at a substitutable position with one or more radicals independently selected from lower alkylsulfinyl, lower alkyl, cyano, carboxyl, lower alkoxycarbonyl, lower haloalkyl, hydroxyl, lower hydroxyalkyl, lower haloalkoxy, amino, lower alkylamino, lower arylamino, lower aminoalkyl, nitro, halo, lower alkoxy, aminosulfonyl, and lower alkylthio; and R⁴ is selected from methyl, hydroxyl and amino; or a pharmaceutically-acceptable salt thereof.

In other embodiments, R¹ is selected from hydroxyl, methyl, ethyl, propyl, isopropyl, butyl, tert-butyl, isobutyl, pentyl, neopentyl, hexyl, carboxyl, carboxypropyl, carboxymethyl, carboxyethyl, benzyl, phenethyl, aminocarbonylmethyl, methoxycarbonylmethyl, methoxycarbonylethyl, methoxymethyl, benzyloxymethyl, phenylethoxymethyl, methylthiomethyl, benzylthiomethyl, N-methylaminomethyl, N,N-dimethylaminomethyl, phenyloxymethyl, phenylthiomethyl, fluoromethyl, difluoromethyl, trifluoromethyl, chloromethyl, dichloromethyl, trichloromethyl, pentafluoroethyl, heptafluoropropyl, fluoromethyl, difluoroethyl, difluoropropyl, dichloroethyl, dichloropropyl, hydroxylmethyl, hydroxylpropyl, hydroxylethyl, cyclohexyl, cyclobutyl, cyclopentyl, cycloheptyl, cyclohexylmethyl, cyclohexylethyl, cyclobutylethyl, cyclopentylmethyl, cycloheptylpropyl, and lower aralkyl selected form benzyl and phenylethyl, wherein the phenyl ring is optionally substituted at a substitutable position with fluoro, chloro, bromo, iodo, methyl, and methoxy; wherein R.sup.3 is selected from phenyl, naphthyl, biphenyl, cyclohexyl, cyclopentyl, cycloheptyl, 1-cyclohexenyl, 2-cyclohexenyl, 3-cyclohexenyl, 4-cyclohexenyl, and 1-cyclopentenyl; R³ is optionally substituted at a substitutable position with one or more radicals independently selected from trifluoromethoxy, N-methylamino, N,N-dimethylamino, N-ethylamino, N,N-dipropylamino, N-butylamino, N-methyl-N-ethylamino, phenylamino, N-methyl-N-phenylamino, methylsulfinyl, ethylsulfinyl, methyl, ethyl, isopropyl, butyl, tert-butyl, isobutyl, pentyl, hexyl, cyano, carboxyl, methoxycarbonyl, fluoromethyl, difluoromethyl, trifluoromethyl, chloromethyl, dichloromethyl, trichloromethyl, pentafluoroethyl, heptafluoropropyl, fluoromethyl, difluoroethyl, difluoropropyl, dichloroethyl, dichloropropyl, hydroxyl, hydroxymethyl, amino, nitro, fluoro, chloro, bromo, iodo, methoxy, ethoxy, propoxy, n-butoxy, pentoxy, hexyloxy, methylenedioxy, aminosulfonyl, methylthio, ethylthio, butylthio, and hexylthio; and R⁴ is selected from methyl, hydroxyl and amino; or a pharmaceutically-acceptable salt thereof.

Useful compounds of Formula II include: 4-[5-ethyl-3-phenylisoxazol-4-yl]benzenesulfonamide; 4-[5-propyl-3-phenylisoxazol-4-yl]benzenesulfonamide; 4-[5-isopropyl-3-phenylisoxazol-4-yl]benzenesulfonamide; 4-[5-butyl-3-phenylisoxazol-4-yl]benzenesulfonamide; 4-[5-isobutyl-3-phenylisoxazol-4-yl]benzenesulfonamide; 4-[5-cyclohexyl-3-phenylisoxazol-4-yl]benzenesulfonamide; 4-[5-neopentyl-3-phenylisoxazol-4-yl]benzenesulfonamide; 4-[5-cyclohexylmethyl-3-phenylisoxazol-4-yl]benzenesulfonamide; 4-[5-(4-chlorophenyl)methyl-3-phenylisoxazol-4-yl]benzenesulfonamide; 4-[5-trifluoromethyl-3-phenylisoxazol-4-yl]benzenesulfonamide; 4-[5-difluoromethyl-3-phenylisoxazol-4-yl]benzenesulfonamide; 4-[5-chloromethyl-3-phenylisoxazol-4-yl]benzenesulfonamide; 4-[5-methyl-3-phenylisoxazol-4-yl]benzenesulfonic acid; 4-[5-propyl-3-phenylisoxazol-4-yl]benzenesulfonic acid; 4-[5-methoxymethyl-3-phenylisoxazol-4-yl]benzenesulfonamide; 4-[5-3-hydroxypropyl)-3-phenylisoxazol-4-yl]benzenesulfonamide; 4-[3-4-chlorophenyl)-5-methyl-isoxazol-4-yl]benzenesulfonamide; 4-[3-4-fluorophenyl)-5-methyl-isoxazol-4-yl]benzenesulfonamide; 4-[3-3-fluoro-4-methylphenyl)-5-methyl-isoxazol-4-yl]benzenesulfonamide; 4-[3-3-aminosulfonyl-4-methoxyphenyl)-5-methyl-isoxazol-4-yl]benzenesulfonamide; 4-[3-3-chloro-4-methylphenyl)-5-methyl-isoxazol-4-yl]benzenesulfonamide; 4-[3-3-fluorophenyl)-5-methyl-isoxazol-4-yl]benzenesulfonamide; 4-[5-hydroxymethyl-3-phenylisoxazol-4-yl]benzenesulfonamide; [4-[4-(aminosulfonyl)phenyl]-3-phenylisoxazol-5-yl]carboxylic acid; 4-[5-hydroxy-3-phenyl-4-isoxazolyl]benzenesulfonamide; 4-[3-methyl-5-phenyl-isoxazol-4-yl]benzenesulfonamide; 4-[5-methyl-3-phenyl-isoxazol-4-yl]benzenesulfonamide; 4-[3-(3-fluoro-4-methoxyphenyl)-5-methyl-isoxazol-4-yl]benzenesulfonamide; [3-(3-chloro-4-methoxyphenyl)-4-[4-(methylsulfonyl)phenyl]isoxazol-5-yl]ace tic acid; 5-methyl-4-[4-(methylsulfonyl)phenyl]-3-phenyl-isoxazole; 3-(3-chloro-4-methoxyphenyl)-5-methyl-4-[4-(methylsulfonyl)phenyl]isoxazole-[4-[4-(aminosulfonyl)phenyl]-3-phenyl-isoxazol-5-yl]acetic acid; [4-[4-(aminosulfonyl)phenyl]-3-phenyl-isoxazol-5-yl]propanoic acid; ethyl [4-[4-(aminosulfonyl)phenyl]-3-phenyl-isoxazol-5-yl]propanoate; [3-(3-fluoro-4-methoxyphenyl)-4-[4-(methylsulfonyl)phenyl]isoxazol-5-yl]acetic acid; [4-[4-(aminosulfonyl)phenyl]-3-(3-fluoro-4-methoxyphenyl)isoxazol-5-yl]propanoic acid; and pharmaceutically-acceptable salts thereof.

The term alkyl, either alone or within other terms such as haloalkyl and alkylsulfonyl, includes linear or branched radicals having one to about twenty carbon atoms or, preferably, one to about twelve carbon atoms. Certain alkyl radicals are “lower alkyl” radicals having one to about ten carbon atoms. Other lower alkyl radicals having one to about five carbon atoms. Examples of such radicals include methyl, ethyl, n-propyl, isopropyl, n-butyl, isobutyl, sec-butyl, tert-butyl, pentyl, iso-amyl, hexyl, octyl and the like. The term hydrido refers to a single hydrogen atom (H). This hydrido radical may be attached, for example, to an oxygen atom to form a hydroxyl radical or two hydrido radicals may be attached to a carbon atom to form a methylene (—CH₂—) radical. The term halo means halogens such as fluorine, chlorine, bromine or iodine atoms. The term haloalkyl refers to radicals wherein any one or more of the alkyl carbon atoms is substituted with halo, e.g., monohaloalkyl, dihaloalkyl and polyhaloalkyl radicals. A monohaloalkyl radical, for example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals. The term hydroxyalkyl includes linear or branched alkyl radicals having one to about ten carbon atoms any one of which may be substituted with one or more hydroxyl radicals. The terms alkoxy and alkoxyalkyl include linear or branched oxy-containing radicals each having alkyl portions of one to about ten carbon atoms, such as methoxy radical. The term alkoxyalkyl also includes alkyl radicals having two or more alkoxy radicals attached to the alkyl radical to form monoalkoxyalkyl and dialkoxyalkyl radicals. The alkoxy or alkoxyalkyl radicals may be further substituted with one or more halo atoms, such as fluoro chloro or bromo to provide haloalkoxy or haloalkoxyalkyl radicals. Examples of alkoxy radicals include methoxy butoxy and trifluoromethoxy. The term aryl, alone or in combination, includes a carbocyclic aromatic system containing one, two or three rings wherein such rings may be attached together in a pendent manner or may be fused. The term aryl includes aromatic radicals such as phenyl, naphthyl, tetrahydronapthyl, indane and biphenyl. The term heterocyclic includes saturated, partially saturated and unsaturated heteroatom-containing ring-shaped radicals, where the heteroatoms may be selected from nitrogen, sulfur and oxygen. Examples of saturated heterocyclic radicals include pyrrolidyl and morpholinyl. The term heteroaryl includes unsaturated heterocyclic radicals. Examples of unsaturated heterocyclic radicals, also termed heteroaryl radicals include: thienyl, pyrryl, furyl, pyridyl, pyrimidyl, pyrazinyl, pyrazolyl, oxazolyl, isoxazolyl, imidazolyl, thiazolyl, pyranyl and tetrazolyl. The term also includes radicals where heterocyclic radicals are fused with aryl radicals. Examples of such fused bicyclic radicals include: benzofuran, benzothiophene, and the like. The term sulfonyl, whether used alone or linked to other terms such as alkylsulfonyl, refers to a divalent radical (—SO₂—). Alkylsulfonyl includes alkyl radicals attached to a sulfonyl radical. The term arylsulfonyl includes sulfonyl radicals substituted with an aryl radical. The terms sulfamyl or sulfonamidyl, whether alone or used with terms such as N-alkylsulfamyl, N-arylsulfamyl, N,N-dialkylsulfamyl and N-alkyl-N-arylsulfamyl, refer to a sulfonyl radical substituted with an amine radical, forming a sulfonamide (—SO₂NH₂). The terms N-alkylsulfamyl and N,N-dialkylsulfamyl refer to sulfamyl radicals substituted, respectively, with one alkyl radical, a cycloalkyl ring, or two alkyl radicals. The terms N-arylsulfamyl and N-alkyl-N-arylsulfamyl refer to sulfamyl radicals substituted, respectively, with one aryl radical, and one alkyl and one aryl radical. The terms carboxy or carboxyl, whether used alone or with other terms, such as carboxyalkyl, refer to —CO₂H. The term carboxyalkyl includes radicals having a carboxy radical attached to an alkyl radical. The term carbonyl, whether used alone or with other terms, such as alkylcarbonyl, denotes —(C═O)—. The term alkylcarbonyl includes radicals having a carbonyl radical substituted with an alkyl radical. An example of an alkylcarbonyl radical is CH₃—(C═O)—. The term alkylcarbonylalkyl, refers an alkyl radical substituted with an alkylcarbonyl radical. The term alkoxycarbonyl means a radical containing an alkoxy radical, as defined above, attached via an oxygen atom to a carbonyl (C═O) radical. Examples of such alkoxycarbonyl radicals include (CH₃)₃ CO—C(═O)— and —(O═)C—OCH₃. The term alkoxycarbonylalkyl includes radicals having alkoxycarbonyl, as defined above substituted to an alkyl radical. Examples of such alkoxycarbonylalkyl radicals include (CH₃)₃ COC(═O)(CH₂)₂—. The term amido when used by itself or with other terms such as amidoalkyl, N-monoalkylamido, N-monoarylamido, N,N-dialkylamido, N-alkyl-N-arylamido, N-alkyl-N-hydroxyamido and N-alkyl-N-hydroxyamidoalkyl, includes a carbonyl radical substituted with an amino radical. The terms N-alkylamido and N,N-dialkylamido refer to amido groups which have been substituted with one alkyl radical and with two alkyl radicals, respectively. The terms N-monoarylamido and N-alkyl-N-arylamido refer to amido radicals substituted, respectively, with one aryl radical, and one alkyl and one aryl radical. The term N-alkyl-N-hydroxyamido includes amido radicals substituted with a hydroxyl radical and with an alkyl radical. The term N-alkyl-N-hydroxyamidoalkyl includes alkyl radicals substituted with an N-alkyl-N-hydroxyamido radical. The term amidoalkyl includes alkyl radicals substituted with amido radicals. The term aminoalkyl includes alkyl radicals substituted with amine radicals. The term alkylaminoalkyl includes aminoalkyl radicals having the nitrogen atom substituted with an alkyl radical. The term amidino refers to an —C(═NH)—NH₂ radical. The term cyanoamidino refers to an —C(═N—CN)—NH₂ radical. The term heterocycloalkyl includes heterocyclic-substituted alkyl radicals such as pyridylmethyl and thienylmethyl. The term aralkyl includes aryl-substituted alkyl radicals such as benzyl, diphenylmethyl, triphenylmethyl, phenethyl, and diphenethyl. The terms benzyl and phenylmethyl are interchangeable. The term cycloalkyl includes radicals having three to ten carbon atoms, such as cyclopropyl cyclobutyl, cyclopentyl, cyclohexyl and cycloheptyl. The term cycloalkenyl includes unsaturated radicals having three to ten carbon atoms, such as cylopropenyl, cyclobutenyl cyclopentenyl cyclohexenyl and cycloheptenyl. The term alkylthio includes radicals containing a linear or branched alkyl radical, of one to ten carbon atoms, attached to a divalent sulfur atom. An example of alkylthio is methylthio, (CH₃—S—). The term alkylsulfinyl includes radicals containing a linear or branched alkyl radical, of one to ten carbon atoms, attached to a divalent —S(═O)— atom. The terms N-alkylamino and N,N-dialkylamino refer to amine groups which have been substituted with one alkyl radical and with two alkyl radicals, respectively. The term acyl, whether used alone, or within a term such as acylamino, refers to a radical provided by the residue after removal of hydroxyl from an organic acid. The term acylamino refers to an amine radical substituted with an acyl group. An example of an acylamino radical is acetylamine (CH₃ C(═O)—NH—).

The term “pharmaceutically-acceptable salts” includes salts commonly used to form alkali metal salts and to form addition salts of free acids or free bases. The nature of the salt is not critical, provided that it is pharmaceutically-acceptable. Suitable pharmaceutically-acceptable acid addition salts of the compounds herein may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, p-hydroxybenzoic, phenylacetic, mandelic, embonic (pamolic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicyclic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine.

The details of one or more embodiments of the invention are set forth in the description below. Other features, objects, and advantages of the invention will be apparent from the description and drawings, and from the claims. The patents, patent applications, and publications referenced herein are hereby incorporated by reference in their entirety.

### DETAILED DESCRIPTION

Celecoxib (CELEBREX®; 4-[[5-(p-tolyl)-3-(trifluoromethyl)-1H-pyrazol-1-yl]]benzenesulfonamide) is described in U.S. Pat. No. 5,466,823 to Talley et al. and valdecoxib (BEXTRA®; 4-(5-methyl-3-phenyl-isoxazol-4-yl)benzenesulfonamide) is described in U.S. Pat. No. 5,633,272 to Talley et al.

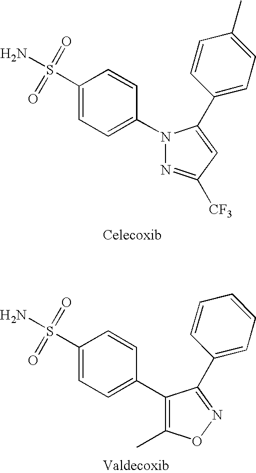

Celecoxib and valdecoxib are considered to be effective inhibitors of COX-2, and both are used for the treatment of inflammation, e.g., inflammation associated with arthritis. Described below are studies demonstrating that celecoxib and valdecoxib are able to inhibit FAAH when present at concentrations above that required for inhibition of COX-2.

FAAH inhibition by celecoxib and valdecoxib was measured and compared to FAAH inhibition both by indomethacin and by URB597 (3′-Carbamoyl-biphenyl-3-yl-cyclohexylcarbamate), a potent FAAH inhibitor (IC₅₀=4.6 nM) that exhibits both anti-nociceptive activity and anxiolytic effects in vivo. The IC₅₀ for COX-1 and for COX-2 inhibition by each compound was also measured.

To assess FAAH inhibition, compounds were tested in an assay using rat brain microsomes and an assay using human brain microsomes using the methods described below. COX-1 inhibition activity and COX-2 inhibition activity were measured in human whole blood as described as below.

### FAAH Rat Brain Membrane (RBM) Homogenate Preparation

Nine adult rats (Charles River CD strain, female, 200 g) were anaesthetized with isofluorane and rapidly decapitated. Each brain was quickly removed and chilled in tubes (3 brains per tube) on ice. Total wet weight of the 9 brains was ˜18 g. 25 mL of homogenization buffer (20 mM HEPES buffer, pH 7.0, with 1 mM MgCl₂) was added to each tube. The brains were homogenized on ice for 1 minute using an Omni GLH homogenizer (Omni International, Marietta, Ga.). The homogenates were transferred to three centrifuge tubes and centrifuged at 36,500 g for 20 minutes at 4° C. The supernatant was discarded and each pellet was re-suspended in 25 ml homogenization buffer. The resuspended material was again centrifuged (36,500g, 20 min at 4° C.). The pellets were combined by resuspension in 10 mL of homogenization buffer and incubated in a 37° C. water bath for 15 min. The tube was then placed on ice for 5 min followed by centrifugation at 36,500 g for 20 minutes at 4° C. The supernatant was discarded and the membrane pellet was then resuspended in 40 mL of resuspension buffer (50 mM Tris-HCl buffer, pH 7.4, containing 1 mM EDTA and 3 mM MgCl₂). A Bradford Protein assay was performed to determine protein concentration. The protein suspension was aliquotted into screw cap Cryo tubes each containing ˜400 μL of suspension, flash frozen in liquid nitrogen and stored at −80° C. until used for the assay.

### FAAH Human Brain Membrane (HBM) Homogenate Preparation

10 g of normal human brain cortex tissue pooled from three donors was purchased from Analytical Biological Services (ABS), Inc. (Wilmington, Del.). The tissue had been collected by ABS, Inc. ˜4 hrs post mortem and was flash frozen and stored at −80° C. The brain tissue was thawed and transferred to a large ceramic mortar on ice. 50 mL of ice-cold homogenization buffer (20 mM HEPES buffer, pH 7.0, with 1 mM MgCl₂) was added to the mortar and the tissue was homogenized with a pestle. The homogenate was centrifuged at 36,500 g for 20 minutes at 4° C. The supernatants were discarded and the pellets re-suspended in homogenization buffer and centrifuged as before. The supernatants were again discarded and the pellets were re-suspended in 30 ml homogenization buffer and incubated in a 37° C. water bath for 20 min. The homogenate was then centrifuged as before. The supernatant was discarded and the membrane pellets were re-suspended in 30 ml resuspension buffer (50 mM Tris-HCl buffer, pH 7.4, containing 1 mM EDTA and 3 mM MgCl₂). A Bradford Protein assay was performed to determine protein concentration. The protein suspension was aliquotted into screw cap Cryo tubes each containing ˜200 μL of suspension, flash frozen in liquid nitrogen and stored at −80° C. until used for the assay.

### Determination of FAAH Activity

FAAH activity was assayed in the respective homogenates (Rat brain or Human brain) using a modification of the method of Omeir et al. (1995 Life Sci 56:1999) and Fowler et al. (1997 J Pharmacol Exp Ther 283:729). To assay FAAH in brain membrane homogenates, 7 μg of RBM protein or 12.5 μg of HBM in 20 μl of 10 mM Tris pH 6.5 was mixed with 180 μl of assay mixture (2.0 μM unlabelled anandamide, 0.03 μCi radiolabeled anandamide [ethanolamine 1-³H] (40-60 Ci/mmol, product number ART-626, American Radiolabelled Chemicals, St. Louis, Mo.), 1 mg/ml Bovine Serum Albumin (fatty acid-free BSA, electrophoresis grade, Sigma, St. Louis Mo.), 10 mM Tris-HCl (pH 6.5), and 1 mM EDTA) in the presence and absence a test compound (in 1% DMSO) and incubated for 10 min at 37° C. Samples were placed on ice to terminate the reactions. The ³H-ethanolamine product and unreacted ³H-anandamide substrate were separated by either chloroform/methanol extraction or by passing the reaction mixture through a glass fiber filter containing activated charcoal. Samples were extracted with chloroform/methanol by adding 0.4 ml of chloroform/methanol (1:1 v/v), vigorously mixing the samples, and separating of the aqueous and organic phases by centrifugation. Radioactivity (corresponding to FAAH-catalyzed breakdown of ³H-anandamide) found in aliquots (0.2 ml) of the aqueous phase was determined by liquid scintillation counting with quench correction. IC₅₀ values are determined as described by Jonsson et al. (2001 Br J Pharmacol 133:1263).

Alternatively, reaction mixtures were purified using a modification of the solid-phase extraction method described by Wilson et al. (2003 Anal Biochem 318: 270). The method of Wilson et al. was modified by acidifying the reaction mixtures by the addition of 10 μl of sodium phosphate solution [0.5M (pH 2.0)] after reactions were incubated at 37° C. for 10 min and chilled on ice. The acidified reaction mixtures were applied to columns of activated charcoal that contained 80 μl of water and was capped with a glass fiber filter. The columns were centrifuged and the eluate was counted as described by Wilson et al.

### Determination of COX-1 activity and COX-2 activity

To measure COX-1 activity in whole blood, 100 μl of whole blood from healthy human donors was combined with a 2 μl aliquot of test compound in vehicle or vehicle alone and incubated for 1 hr at 37° C. as described by Berg et al. (1999 Inflamm Res 48, 369-379). Serum was isolated from the sample by centrifugation at 12,000 g for 5 min at 4° C. and was assayed for thromboxane B2 (TXB2) levels using an ELISA assay (e.g., Cayman EIA Kit, Catalog Number 519031). To measure COX-2 activity in whole blood, 100 μl of heparinized whole blood from healthy human donors was combined with a 1 μl aliquot of 10 μg/ml LPS (lipopolysaccharide) and a 2 μl aliquot of test compound in vehicle or vehicle alone and incubated for 24 h at 37° C. as described by Berg et al. (supra). Serum was isolated from the sample by centrifugation at 12,000 g for 5 min at 4° C. and assayed for PGE₂ using an ELISA assay (e.g., Cayman EIA Kit, Catalog Number 514010).

Results

The table below shows that celecoxib and valdecoxib can inhibit FAAH. The results are particularly striking for human, as opposed to rat, FAAH. This is in contrast to the results obtained for indomethacin which is significantly more potent in the RBM assay relative to the HBM assay. These results show that celecoxib and valdecoxib can be used to inhibit FAAH when administered at concentrations that are above that which inhibit COX-2. Rofecoxib, a potent COX-2 selective drug, is a relatively weak inhibitor of FAAH from either rat or human sources. Previous studies have focused solely on activity of these compounds against the rat enzyme (yielding similar results) but have not characterized inhibition of the human FAAH enzyme (Fowler et al, 2003, J Enzyme Inhib Med Chem, 18(1):55-58).

| | | | | IC₅₀ FAAH |
|---|---|---|---|---|
| | | IC₅₀ | IC₅₀ FAAH | (μM) |
| | IC₅₀ COX-1 | COX-2 | (μM) | Human |
| Compound | (μM) | (μM) | Rat Brain | Brain |
| Indomethacin | 0.18 +/− 0.05 | 0.23 +/− | 52 +/− 39 | 96 +/− 27 |
| | | 0.03 | | |
| Celecoxib | 12.08 +/− 0.75 | 0.42 +/− | 167 +/− 95 | 39 +/− 14 |
| | | 0.02 | | |
| Valdecoxib | 100 | 0.15 | 280 +/− 204 | 41 +/− 24 |
| Rofecoxib | 39 | 0.24 | 452 +/− 110 | 198 +/− 89 |
| URB597 | N.D. | N.D. | 0.02 | 0.06 |

Additional Compounds

Compounds that are structurally related to celecoxib, for example compounds described in U.S. Pat. No. 5,466,823 (hereby incorporated by reference), may also be useful for inhibiting FAAH. Thus, compounds having Formula I, below, may be useful. 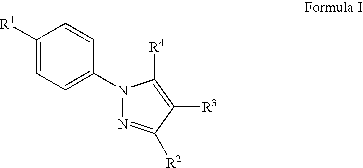
wherein: R¹ is sulfamyl; wherein R² is haloalkyl; wherein R³ is selected from hydrido, and alkyl; and wherein R⁴ is selected from aryl, cycloalkyl, and cycloalkenyl; wherein R⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy, hydroxyalkyl, haloalkoxy, sulfamyl, N-alkylsulfamyl, amino, N-alkylamino, N,N-dialkylamino, heterocyclic, nitro and acylamino; and pharmaceutically-acceptable salts thereof. In certain embodiments, the compounds used in the method have Formula I, provided that the compound is not celecoxib.

Compounds that are structurally related to valdecoxib, for example compounds described in U.S. Pat. No. 5,633,272 (hereby incorporated by reference), may also be useful for inhibiting FAAH. Thus, compounds having Formula II or Formula IIA below may be useful. 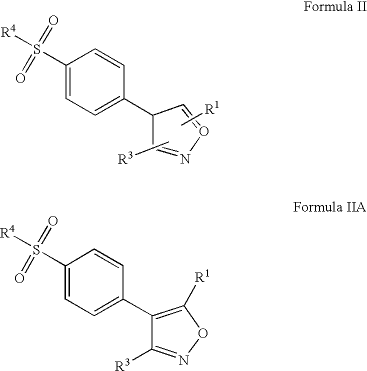
wherein: R¹ is selected from alkyl, carboxyalkyl, alkoxycarbonyl, aminocarbonyl, aminocarbonylalkyl, alkoxycarbonylalkyl, carboxyl, alkoxy, haloalkoxy, aralkoxy, cycloalkylalkoxy, alkylthio, aralkylthio, cycloalkylalkylthio, alkoxyalkyl, aralkoxyalkyl, alkylthioalkyl, aralkylthioalkyl, alkylaminoalkyl, aryloxyalkyl, arylthioalkyl, hydroxyl, amino, hydroxyalkyl, haloalkyl, cycloalkyl, cycloalkylalkyl, aralkyl, halo, alkylamino, aralkylamino, N-alkyl-N-aralkylamino, N-alkyl-N-cycloalkylalkylamino, arylcarbonyloxyalkyl, arylcarbonylthio, alkoxycarbonyloxyalkyl, alkylaminocarbonyloxyalkyl, alkoxycarbonylthioalkyl, and alkylaminocarbonylthioalkyl; R³ is selected from cycloalkyl, cycloalkenyl, and aryl, wherein R³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and R⁴ is selected from lower alkyl, hydroxyl, and amino; and pharmaceutically-acceptable salts thereof. In certain embodiments, the compounds used in the method have Formula II or Ia, provided that the compound is not valdeecoxib.
Methods for Assessing Activity in Vitro and in Vivo

The ability of a compound to inhibit FAAH can be measured as described above or using any of the assays described below.

### FAAH Human Carcinoma Cell Membrane (HCM) Homogenate Preparation

Human breast epithelial carcinoma MCF7 cells are obtained from the American Type Culture Collection (ATCC Number HTB-22, Manassas, Va.) and cultured as essentially as described by ATCC. Briefly, cells are grown in Eagle's Minimum Essential Medium (ATCC catalog no. 30-2003) supplemented with 4 mM L-glutamine, 10% final volume of fetal bovine serum (ATCC catalog no. 30-2020), and 0.1 mg/mL human recombinant insulin (Sigma, St. Louis, Mo.). The cells are grown in 5% carbon dioxide in air. When cells reach ˜80% confluency, adherent cells are rinsed with Hanks Balanced Salts Solution (ATCC catalog no. 30-2213), scraped into suspension and collected by centrifugation in a clinical centrifuge at room temperature. Cell pellets are then washed by resuspension in Hanks Balanced Salts Solution followed by centrifugation. Cell pellets are then flash frozen in a dry ice and ethanol bath and stored at −80 ° C. Cell pellets are thawed and 25 mL of homogenization buffer is added. Membrane homogenates of MCF7 cells are then prepared as described above for rat brain homogenates. A Bradford Protein assay is performed to determine the protein concentration. The protein is aliquotted into screw cap Cryo tubes each containing ˜200 μL, flash frozen in liquid nitrogen and stored at −80° C. until used for the assay. In vitro FAAH activity can be determined as described above for RBM and HBM preparations with modifications as necessary to accommodate the specific activity of each extract.

Whole Cell Anandamide Hydrolysis Assay

FAAH activity can also be assayed in whole cells using methods disclosed previously (Maccarone et al. 1998 J Biol Chem 273:32332 and Bisogno et al. 1997 J Biol Chem 272 :3315). In addition to the cell lines described in Maccarone et al. and Bisogno et al., MCF7 (ATCC designation HTB-22) and T84 (ATCC designation CCL-248) cell lines may be used in these assays.

Therapeutic Methods

Glaucoma and Ocular Disorders

The compounds can be used to prevent and/or treat glaucoma and other disorders characterized by ocular hypertension.

Sleep Disorders

The compounds can be used to prevent and/or treat a sleep disorder that affects the subject's ability to fall asleep and/or remain asleep, and/or results in unrefreshing sleep. The term “sleep disorder” includes insomnia, night terrors, bruxism, somnambulism, sleep apnea, restless leg syndrome, unrefreshing sleep, seasonal affective disorder, circadian rhythm adjustment disorders, and the like.

Insomnia is typically classed into sleep onset insomnia, where a subject takes more than 30 minutes to fall asleep; and sleep maintenance insomnia, where the subject spends more than 30 minutes awake during an expected sleep period, or, for example, waking before the desired wake-up time with an inability to get back to sleep. Sleep disorders include both endogenous disorders, such as sleep apnea, and disorders related to behavioral or external environmental factors. For example, sleep disorders include a subject's difficulty in adjusting to a new circadian rhythm, for example, due to jet lag; night, extended, or irregular work shifts; and the like. A sleep disorder can also arise in a subject that has other disorders, diseases, or injuries, or in a subject being treated with other medications, where the subject as a result has difficulty falling asleep and/or remaining asleep, or experiences unrefreshing sleep. For example, the disclosed method is useful for inducing sleep in a subject having difficulty sleeping as the result of undergoing chemotherapy, or as a result of injuries, or as the result of stress or mood disorders such as depression, anxiety, and the like.

Sleep disorders include conditions recognized by one skilled in the art as sleep disorders, for example, conditions known in the art or conditions which are proposed to be sleep disorders or discovered to be sleep disorders. See, for example, Thorpy, M J International Classification of Sleep Disorders, Revised: Diagnostic and Coding Manual. American Sleep Disorders Association; Rochester, Minnesota 1997; and JCD CM, International Classification of Diseases, Ninth Revision, Clinical Modification, National Center for Health Statistics, Hyattsville, Md.

Sleep disorders can be generally classed into dyssomnias, e.g., intrinsic, extrinsic, and circadian rhythm disorders; parasomnias, e.g., arousal, sleep-wake transition, and rapid eye movement (REM) associated disorders, and other parasomnias; disorders associated with mental, neurological, and other medical disorders; and other sleep disorders.

Intrinsic sleep disorders include, for example, psychophysiological insomnia, sleep state misperception, idiopathic insomnia, narcolepsy, recurrent hypersomnia, idiopathic hypersomnia, post-traumatic hypersomnia, obstructive sleep apnea syndrome, central sleep apnea syndrome, central alveolar hypoventilation syndrome, periodic limb movement disorder, restless legs syndrome, and the like.

Extrinsic sleep disorders include, for example, inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating (drinking) syndrome, hypnotic-dependent sleep disorder, stimulant-dependent sleep disorder, alcohol-dependent sleep disorder, toxin-induced sleep disorder, and the like.

Circadian rhythm sleep disorders include, for example, time-zone change (jet lag) syndrome, shift work sleep disorder, irregular sleep-wake pattern, delayed sleep phase syndrome, advanced sleep phase syndrome, non 24h sleep-wake disorder, and the like.

Arousal sleep disorders include, for example, confusional arousals, sleepwalking, sleep terrors, and the like.

Sleep-wake transition disorders include, for example, rhythmic movement disorder, sleep starts, sleeptalking, nocturnal leg cramps, and the like.

REM-associated sleep disorders include, for example, nightmares, sleep paralysis, impaired sleep-related penile erections, sleep-related painful erections, REM sleep-related sinus arrest, REM sleep behavior disorders, and the like.

Other parasomnias include, for example, sleep bruxism, sleep enuresis, sleep-related abnormal swallowing syndrome, nocturnal paroxysmal dystonia, sudden unexplained nocturnal death syndrome, primary snoring, infant sleep apnea, congenital central hypoventilation syndrome, sudden infant death syndrome, benign neonatal sleep myoclonus, and the like. A “sleep disorder” may also arise in a subject that has other medical disorders, diseases, or injuries, or in a subject being treated with other medications or medical treatments, where the subject as a result has difficulty falling asleep and/or remaining asleep, or experiences unrefreshing sleep, e.g., the subject experiences sleep deprivation. For example, some subjects have difficulty sleeping after undergoing medical treatment for other conditions, e.g., chemotherapy or surgery, or as a result of pain or other effects of physical injuries.

It is well known in the art that certain medical disorders, for example, central nervous system (CNS) disorders, e.g., mental or neurological disorders, e.g., anxiety, can have a sleep disorder component, e.g., sleep deprivation. Thus, treating a sleep disorder also includes treating a sleep disorder component of other disorders, e.g., CNS disorders. Further, treating the sleep disorder component of CNS disorders can also have the beneficial effect of ameliorating other symptoms associated with the disorder. For example, in some subjects experiencing anxiety coupled with sleep deprivation, treating the sleep deprivation component also treats the anxiety component. Thus, the present invention also includes a method of treating such medical disorders.

Sleep disorders associated with mental disorders include psychoses, mood disorders, anxiety disorders, panic disorder, addictions, and the like. Specific mental disorders include, for example, depression, obsessive compulsive disorder, affective neurosis/disorder, depressive neurosis/disorder, anxiety neurosis, dysthymic disorder, behavior disorder, mood disorder, schizophrenia, manic depression, delirium, alcoholism, and the like.

Sleep disorders associated with neurological disorders include, for example, cerebral degenerative disorders, dementia, parkinsonism, fatal familial insomnia, sleep related epilepsy, electrical status epilepticus of sleep, sleep-related headaches, and the like. Sleep disorders associated with other medical disorders include, for example, sleeping sickness, nocturnal cardiac ischemia, chronic obstructive pulmonary disease, sleep-related asthma, sleep-related gastroesophageal reflux, peptic ulcer disease, fibrositis syndrome, and the like.

In some circumstances, sleep disorders are also associated with pain, e.g., neuropathic pain associated with restless leg syndrome; migraine; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labor pain; cancer pain; postchemotherapy pain; post-stroke pain; post-operative pain; neuralgia; conditions associated with visceral pain including irritable bowel syndrome, migraine and angina; and the like.

Other sleep disorders include, for example, short sleeper, long sleeper, subwakefulness syndrome, fragmentary myoclonus, sleep hyperhidrosis, menstrual associated sleep disorder, pregnancy-associated sleep disorder, terrifying hypnagogic hallucinations, sleep-related neurogenic tachypnea, sleep-related laryngospasm, sleep choking syndrome, and the like.

Insomnia is typically classed into sleep onset insomnia, where a subject takes more than 30 minutes to fall asleep; and sleep maintenance insomnia, where the subject spends more than 30 minutes awake during an expected sleep period, or, for example, waking before the desired wake-up time with difficulty or an inability to get back to sleep. Some of the disclosed compounds are effective in treating sleep onset and sleep maintenance insomnias, insomnia resulting from circadian rhythm adjustment disorders, or insomnia resulting from CNS disorders. In one embodiment, a subject is treated for a circadian rhythm adjustment disorder. In another embodiment a subject is treated for insomnia resulting from a mood disorder. In other embodiments, a subject is treated for sleep apnea, somnambulism, night terrors, restless leg syndrome, sleep onset insomnia, and sleep maintenance insomnia. In other embodiments, a subject is treated for, sleep onset insomnia or sleep maintenance insomnia.

Compounds of the invention can be used to for inducing, prolonging and/or enhancing sleep. This can encompass the treatment of a sleep disorder, i.e., a difficulty in achieving satisfactory sleep due to some internal or external factor, e.g. pain, stress or anxiety, misuse of stimulants or depressants, or temporary disturbance of lifestyle and it can encompass elective desires on the part of a user to achieve a particularly beneficial period of sleep. Such a desire may, for instance, arise in anticipation of important events the following day or in the near future for which a person may wish to be fully alert and refreshed.

The compounds can help achieve any of the following goals: getting to sleep, especially stage 1 sleep; staying asleep; sleeping well; waking refreshed; waking alert; faster onset to stage 1 sleep; increasing duration of sleep periods; decreasing the number and duration of awakenings; increasing total duration of sleep; increasing probability of sleeping well; reducing insomnia, especially chronic or mild-moderate insomnia; decreasing disturbances during sleeptime; and improving quality of sleep. Meeting these goals can be determined by any standard or, known subjective or objective measures, for instance the Karolinska scale, Loughborough sleep log or actimetry.

Improved sleep can assist in keeping awake; keeping alert; keeping refreshed; and performing well the next day

An effective amount of a compound of the disclosed invention is the quantity which, when administered to a subject in need of treatment, results in the subject falling asleep more rapidly, results in more refreshing sleep, reduces duration or frequency of waking during a sleep period, or reduces the duration, frequency, or intensity of episodes of night terrors, bruxism, or somnambulism. The amount of the disclosed compound to be administered to a subject will depend on the particular disorder, the mode of administration, co-administered compounds, if any, and the characteristics of the subject, such as general health, other diseases, age, sex, genotype, body weight and tolerance to drugs. The skilled artisan will be able to determine appropriate dosages depending on these and other factors.

The degree of refreshedness and quality of sleep may be determined by the “morning” log of the Loughborough sleep log with the highest degree of refreshedness or quality of sleep being represented as 1 and the lowest being represented as 5. Accordingly, the percentage increase in refreshedness or quality of sleep is measured in this context by the decrease in the mean refreshedness or quality of sleep.

The response of feeling extremely alert, very alert or alert can be determined, for instance, by the Karolinska 9-point scale.

Other measures of sleep parameters include the sleep disturbance index (SDI) and time to sleep onset (TTSO) that can both be measured by actimetry.

The compounds can be used in combination with therapies currently used for the treatment of sleep disorders, e.g., Aldesleukin (Proleukin), Amantadine (Symmetrel), Baclofen (Lioresal), Bepridil (Vascor), Carisoprodol (Soma), Clonazepam (Klonopin), Diazepam (Valium), Diphenhydramine (Sominex, Nytol), Doxylamine (Unisom), Estazolam (ProSom), Flurazepam (Dalmane), Gabapentin, Lorazepam (Ativan), Levodopa-carbidopa (Sinemet), Melatonin, Methylphenidate (Ritalin), Modanfinil (Provigil), Pemoline (Cylert), Pergolide, Pramipexole, Promethazine (Phenergan), Quazepam (Doral), Rimantadine (Flumadine), Sibutramine (Meridia), Sodium oxybate, Synthetic conjugated estrogens (Cenestin), Temazepam (Restoril), Triazolam (Halcion), Zaleplon (Sonata), and Zolpidem (Ambien).

Obesity Related Disorders

The compounds may be used to treat obesity and/or to reduce or control body weight (or fat) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). Obesity is a condition in which there is an excess of body fat. In many cases, an individual is considered obese if the individual has a body mass index (BMA) greater than or equal to 30 kg/m² or if the individual has at least one co-morbidity and a BMI greater than or equal to 27 kg/m². In certain situations, a subject at risk for obesity is an otherwise healthy subject with a BMI of 25 kg/m² to less than 30 kg/m² or a subject with at least one co-morbidity with a BMI of 25 kg/m² to less than 27 kg/M².

The increased risks associated with obesity is thought to occur at a lower BMI in Asians. In some situations, obesity in an Asian refers to a condition whereby a subject with at least one obesity-induced or obesity-related co-morbidity that requires weight reduction or that would be improved by weight reduction, has a BMI greater than or equal to 25 kg/m². In Asians, an obese subject sometimes refers to a subject with at least one obesity-induced or obesity-related co-morbidity that requires weight reduction or that would be improved by weight reduction, with a BMI greater than or equal to 25 kg/m². In some situations, an Asian at risk of obesity is a subject with a BMI of greater than 23 kg/M² to less than 25 kg/M².

Obesity-induced or obesity-related co-morbidities include, but are not limited to, diabetes, noninsulin dependent diabetes mellitus type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver, cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

Obesity-related disorders are disorders that are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrhythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. The compounds of the invention may be used to reduce or control body weight (or fat) or to prevent and/or treat obesity or other appetite related disorders related to the excess consumption of food, ethanol and other appetizing substances. The compounds may be used to modulate lipid metabolism, reduce body fat (e.g. via increasing fat utilization) or reduce (or suppress) appetite (e.g. via inducing satiety).

Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastroesophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The compounds can be administered in combination with anti-obesity agents, including, but not limited to:

11βHSD-1 (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO01/90091, WO01/90090, WO01/90092 and WO02/072084;

5HT (serotonin) transporter inhibitors, such as paroxetine, fluoxetine, fenfluramine, fluvoxamine, sertraline, and imipramine, and those disclosed in WO03/00663;

5HT antagonists such as those in WO03/037871, WO03/037887, and the like;

5HT1a modulators such as those disclosed in WO03/031439, and the like;

5HT-2 agonists;

5HT2c (serotonin receptor 2c) agonists, such as BVT933, DPCA37215, IK264, PNU 22394, WAY161503, R-1065, and YM 348 and those disclosed in U.S. Pat. No. 3,914,250 and PCT publication Nos. WO02/36596, WO02/48124, WO02/10169, WO01/66548, WO02/44152, WO02/51844, WO02/40456, and WO02/40457;

5HT6 receptor modulators, such as those in WO03/030901, WO03/035061, WO03/039547, and the like;

ACC2 (acetyl-CoA carboxylase-2) inhibitors;

acyl-estrogens, such as oleoyl-estrone, disclosed in del Mar-Grasa, M. et al., Obesity Research, 9:202-9 (2001) and Japanese Patent Application No. JP 2000256190;

alpha-lipoic acid (alpha-LA);

anorectic bicyclic compounds such as 1426 (Aventis) and 1954 (Aventis), and the compounds disclosed in WO00/18749, WO01/32638, WO01/62746, WO01/62747, and WO03/015769;

AOD9604;

appetite suppressants such as those in WO03/40107;

ATL-962 (Alizyme PLC);

benzocaine;

benzphetamine hydrochloride (Didrex);

bladderwrack (focus vesiculosus);

BRS3 (bombesin receptor subtype 3) agonists;

bupropion;

caffeine;

CB1 (cannabinoid-1 receptor) antagonist/inverse agonists, such as rimonabant (Acomplia; Sanofi Synthelabo), SR-147778 (Sanofi Synthelabo), BAY 65-2520 (Bayer), and SLV 319 (Solvay), and those disclosed in U.S. Pat. Nos. 4,973,587, 5,013,837, 5,081,122, 5,112,820, 5,292,736, 5,532,237, 5,624,941, 6,028,084, and 6,509,367 and WO96/33159, WO97/29079, WO98/31227, WO98/33765, WO98/37061, WO98/41519, WO98/43635, WO98/43636, WO99/02499, WO00/10967, WO0/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and U.S. Pat. No. 6,509,367 and EPO Application No. EP-658546;

CCK agonists;

CCK-A (cholecystokinin-A) agonists, such as AR-R 15849, GI 181771, JMV-180, A-71378, A-71623 and SR146131, and those described in U.S. Pat. No. 5,739,106;

chitosan;

chromium;

CNTF (Ciliaryneurotrophic factors), such as GI-181771 (Glaxo-SmithKline), SR146131 (Sanofi Synthelabo), butabindide, PD170,292, and PD 149164 (Pfizer);

CNTF derivatives, such as axokine (Regeneron), and those disclosed in PCT Application Nos. WO 94/09134, WO 98/22128, and WO 99/43813;

conjugated linoleic acid;

corticotropin-releasing hormone agonists;

dehydroepiandrosterone;

DGAT1 (diacylglycerol acyltransferase 1) inhibitors;

DGAT2 (diacylglycerol acyltransferase 2) inhibitors;

dicarboxylate transporter inhibitors;

diethylpropion hydrochloride (Tenuate);

dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444 and the compounds disclosed in PCT publication Nos. WO02/083128, WO02/062764, WO03/000180, WO03/000181, WO03/000250, WO03/002530, WO03/002531, WO03/002553, WO03/002593, WO03/004498, WO03/004496,WO03/017936, WO03/024942, WO03/024965, WO03/033524, WO03/037327 and EP1258476;

ephedra;

exendin-4 (an inhibitor of glp-1)

FAS (fatty acid synthase) inhibitors, such as Cerulenin and C75;

fat resorption inhibitors such as those in WO03/05345 1, and the like;

fatty acid transporter inhibitors;

fiber (psyllium, plantago, guar fiber);

galanin antagonists;

galega (Goat's Rue, French Lilac);

garcinia cambogia;

germander (teucrium chamaedrys);

ghrelin antagonists, such as those disclosed in PCT Application Nos. WO 01/87335, and WO 02/08250;

GLP-1 (glucagon-like peptide 1) agonists (e.g. exendin-4);

glp-1 (glucagon-like peptide-1);

glucocorticoid antagonists;

glucose transporter inhibitors;

growth hormone secretagogue receptor agonists/antagonists, such as NN703, hexarelin, MK-0677, SM-130686, CP-424,391, L-692,429 and L-163,255, and such as those disclosed in U.S. Pat. No. 6,358,951, U.S. Patent Application Nos. 2002/049196 and 2002/022637, and PCT Application Nos. WO 01/56592 and WO 02/32888; growth hormone secretagogues, such as those disclosed and specifically described in U.S. Pat. No. 5,536,716;

H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in PCT publication No. WO02/15905 and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO03/024928 and WO03/024929;

interleukin-6 (IL-6) and modulators thereof, as in WO03/057237, and the like;

L-camitine;

leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; leptin, including recombinant human leptin (PEG-OB, Hoffman La Roche) and recombinant methionyl human leptin (Amgen);

lipase inhibitors, such as tetrahydrolipstatin (orlistat/Xenical®), Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in PCT publication No. WO01/77094, and U.S. Pat. Nos. 4,598,089, 4,452,813, 5,512,565, 5,391,571, 5,602,151, 4,405,644, 4,189,438, and 4,242,453;

lipid metabolism modulators such as maslinic acid, erythrodiol, ursolic acid uvaol, betulinic acid, betulin, and the like and compounds disclosed in WO03/011267;

Mc3r (melanocortin 3 receptor) agonists;

Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410;

Mc5r (melanocortin 5 receptor) modulators, such as those disclosed in WO97/19952, WO00/15826, WO00/15790, US 20030092041;

MCH2R (melanin concentrating hormone 2R) agonist/antagonists;

melanin concentrating hormone antagonists;

melanin-concentrating hormone 1 receptor (MCHR) antagonists, such as T-226296 (Takeda), SNP-7941 (Synaptic), and those disclosed WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480 and Japanese Patent Application Nos. JP 13226269, and JP1437059;

melanocortin agonists, such as Melanotan II or those described in WO 99/64002 and WO 00/74679;

Metformin (Glucophage®);

mGluR5 modulators such as those disclosed in WO03/029210, WO03/047581, WO03/048137, WO03/051315, WO03/051833, WO03/053922, WO03/059904, and the like;

monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO01/27068, and WO01/62341;

NE (norepinephrine) transport inhibitors, such as GW 320659, despiramine, talsupram, and nomifensine;

nomame herba;

non-selective serotonin/norepinephrine transport inhibitors, such as sibutramine or fenfluramine; NPY 1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528;

NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683, European Patent Nos. EP-01010691, and EP-01044970 and PCT Publication Nos. WO97/19682, WO97/20820, WO97/20821, WO97/20822, WO97/20823, WO98/27063, WO0/107409, WO00/185714, WO00/185730, WO00/64880, WO00/68197, WO00/69849, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al., J. Med. Chem. 43:4288-4312 (2000);

opioid antagonists, such as nalmefene (Revex®), 3-methoxynaltrexone, naloxone, and naltrexone and those disclosed in WO00/21509;

orexin antagonists, such as SB-334867-A and those disclosed in PCT publication Nos. WO01/96302, WO01/68609, WO02/44172, WO02/51232, WO02/51838, WO02/089800, WO02/090355, WO03/023561, WO03/032991, and WO03/037847;

PDE (phosphodiesterase) inhibitors, such as theophylline, pentoxifylline, zaprinast, sildenafil, amrinone, milrinone, cilostamide, rolipram, and cilomilast; peptide YY and fragments and variants thereof (e.g. YY₃₋₃₆ (PYY₃₋₃₆)(N. Engl. J. Med. 349:941, 2003; ikpeapge daspeelnry yaslrhylnl vtrqry) and PYY agonists such as those disclosed in WO03/026591;

phendimetrazine;

phentermine,

phosphate transporter inhibitors;

phosphodiesterase-3B (PDE3B) inhibitors;

phytopharm compound 57 (CP 644,673);

pyruvate;

SCD-1 (stearoyl-CoA desaturase-1) inhibitors;

serotonin reuptake inhibitors, such as dexfenfluramine, fluoxetine, and those in U.S. Pat. No. 6,365,633, and WO01/27060, and WO01/162341;

T71 (Tularik; Inc.; Boulder CO);

thyroid hormone β agonists, such as KB-2611 (KaroBioBMS), and those disclosed in WO02/15845 and Japanese Patent Application No. JP 2000256190;

Topiramate (Topimax®);

transcription factor modulators such as those disclosed in WO03/026576;

UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propeny-1]benzoic acid (TTNPB), retinoic acid, and those disclosed in PCT Patent Application No. WO 99/00123;

β3 (beta adrenergic receptor 3) agonists, such as AD9677/TAK677 (Dainippon/Takeda), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), and SR 59119A, and those disclosed in U.S. Pat. No. 5,705,515, U.S. Pat. No. 5,451,677 and PCT publication Nos. WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881;

β-hydroxy steroid dehydrogenase-1 inhibitors (β-HSD-1);

β-hydroxy-β-methylbutyrate;

Anxiety Related Disorders

The compounds of the invention can also be used to treat anxiety disorder (including generalized anxiety disorder, panic disorder, and social anxiety disorder) and depression. Anxiety disorders are a group of psychological problems whose key features include excessive anxiety, fear, worry, avoidance, and compulsive rituals, and produce or result in inordinate morbidity, over utilization of healthcare services, and functional impairment. They are among the most prevalent psychiatric conditions in the United States and in most other countries. Anxiety disorders listed in the Diagnostic and Statistical Manual of Mental Disorders (Fourth Edition, Revised 1994, published by the American Psychiatric Association, Washington, D.C., pages 393-444) include panic disorder with and without agoraphobia, agoraphobia without history of panic disorder, specific phobia, social phobia, obsessive-compulsive disorder (OCD), post-traumatic stress disorder (PTSD), acute stress disorder, generalized anxiety disorder (GAD), anxiety disorder due to a general medical condition, substance-induced anxiety disorder, specific phobia, and anxiety disorder not otherwise specified.

Obsessive compulsive disorder is characterized by recurrent and persistent ideas, thoughts, impulses or images (obsessions) that are ego-dystonic and/or repetitive, purposeful and intentional behaviors (compulsions) that are recognized by the person as excessive or unreasonable. The obsessions or compulsions cause marked distress, are time-consuming, and/or significantly interfere with social or occupational functioning.

Panic disorder is characterized by recurrent unexpected panic attacks and associated concern about having additional attacks, worry about the implications or consequences of the attacks, and/or a significant change in behavior related to the attacks. A panic attack is defined as a discrete period of intense fear or discomfort in which four (or more) of the following symptoms develop abruptly and reach a peak within 10 minutes: (1) palpitations, pounding heart, or accelerated heart rate; (2) sweating; (3) trembling or shaking; (4) sensations of shortness of breath or smothering; (5) feeling of choking; (6) chest pain or discomfort; (7) nausea or abdominal distress; (8) feeling dizzy unsteady, lightheaded, or faint; (9) derealization (feelings of unreality) or depersonalization (being detached from oneself); (10) fear of losing control; (11) fear of dying; (12) paresthesias (numbness or tingling sensations); and (13) chills or hot flushes. Panic disorder may or may not be associated with agoraphobia, or an irrational and often disabling fear of being out in public.

Social anxiety disorder, also known as social phobia, is characterized by a marked and persistent fear of one or more social or performance situations in which the person is exposed to unfamiliar people or to possible scrutiny by others. Exposure to the feared situation almost invariably provokes anxiety, which may approach the intensity of a panic attack. The feared situations are avoided or endured with intense anxiety or distress. The avoidance, anxious anticipation, or distress in the feared situation(s) interferes significantly with the person's normal routine, occupational or academic functioning, or social activities or relationships, or there is marked distress about having the phobias.

Lesser degrees of performance anxiety or shyness generally do not require psychopharmacological treatment.

Generalized anxiety disorder is characterized by excessive anxiety and worry (apprehensive expectation) that is persistent for at least 6 months and which the person finds difficult to control. It must be associated with at least 3 of the following 6 symptoms: restlessness or feeling keyed up or on edge, being easily fatigued, difficulty concentrating or mind going blank, irritability, muscle tension, and sleep disturbance. The diagnostic criteria for this disorder are described in further detail in DSM-IV, which is incorporated herein by reference (American Psychiatric Association, 1994).

Post-traumatic stress disorder (PTSD), as defined by DSMIII-R/IV, requires exposure to a traumatic event that involved actual or threatened death or serious injury, or threat to the physical integrity of self or others, and a response which involves intense fear, helplessness, or horror. Symptoms that occur as a result of exposure to the traumatic event include re-experiencing of the event in the form of intrusive thoughts, flashbacks or dreams, and intense psychological distress and physiological reactivity on exposure to cues to the event; avoidance of situations reminiscent of the traumatic event, inability to recall details of the event, and/or numbing of general responsiveness manifested as diminished interest in significant activities, estrangement from others, restricted range of affect, or sense of foreshortened future; and symptoms of autonomic arousal including hypervigilance, exaggerated startle response, sleep disturbance, impaired concentration, and irritability or outbursts of anger. A PTSD diagnosis requires that the symptoms are present for at least a month and that they cause clinically significant distress or impairment in social, occupational, or other important areas of functioning.

It is contemplated that the compounds will be effective in treating obsessions and compulsions in patients who have been diagnosed as having obsessive compulsive disorder based upon administration of appropriate tests, which may include, but are not limited to any of the following: Yale Brown Obsessive Compulsive Scale (YBOCS) (for adults), National Institute of Mental Health Global OCD Scale (NIMH GOCS), and CGI-Severity of Illness scale. It is further contemplated that the compounds will be effective in inducing improvements in certain of the factors measured in these tests, such as a reduction of several points in the YBOCS total score. It is also contemplated that the compounds of this invention will be effective in preventing relapse of obsessive-compulsive disorder.

The invention provides a method of treating obsessions and compulsions in a subject with obsessive-compulsive disorder, which comprises administering to the subject an amount of any of the compounds of the invention effective to treat the subject's obsessions and compulsions.

It is contemplated that the compounds will be effective in treating panic disorder in patients who have been diagnosed with panic disorder on the basis of frequency of occurrence of panic attacks, or by means of the CGI-Severity of Illness scale. It is further contemplated that the compounds of the invention will be effective in inducing improvements in certain of the factors measured in these evaluations, such as a reduction in frequency or elimination of panic attacks an improvement in the CGI-Severity of Illness scale or a CGI Global Improvement score of 1 (very much improved), 2 (much improved) or 3 (minimally improved). It is also contemplated that the compounds of this invention will be effective in-preventing relapse of panic disorder.

It is contemplated that the compounds will be effective in treating social anxiety disorder in patients who have been diagnosed as having social anxiety disorder based upon the administration of any of the following tests: the Liebowitz Social Anxiety Scale (LSAS), the CGI-Severity of Illness scale, the Hamilton Rating Scale for Anxiety (HAM-A), the Hamilton Rating Scale for Depression (HAM-D), the axis V Social and occupational Functioning Assessment Scale of DSM-IV, the axis II (ICD10) World—Health organization Disability Assessment, Schedule 2 (DAS-2), the Sheehan Disability Scales, the Schneier Disability Profile, the World Health Organization Quality of Life-100 (WHOQOL-100)), or other tests as described in Ballenger, J C et al, 1998, J Clin Psychiatry 59 Suppl 17:54-60., which is incorporated herein by reference. It is further contemplated that the compounds of the invention will be effective in inducing improvements as measured by these tests, such as the a change from baseline in the Liebowitz Social Anxiety Scale (LSAS), or a CGI-Global Improvement score of 1 (very much improved), 2 (much improved) or 3 (minimally improved). It is also contemplated that the compounds of this invention will be effective in preventing relapse of social anxiety disorder.

It is contemplated that the compounds will be effective in treating generalized anxiety disorder in patients who have been diagnosed as having this disorder based upon the diagnostic criteria described in DSM-IV. It is further contemplated that the compounds of the invention will be effective in reducing symptoms of this disorder, such as the following: excessive worry and anxiety, difficulty controlling worry, restlessness or feeling keyed up or on edge, being easily fatigued, difficulty concentrating or mind going blank, irritability, muscle tension, or sleep disturbance. It is also contemplated that the compounds of this invention will be effective in preventing relapse of general anxiety disorder.

It is contemplated that the compounds will be effective in treating PTSD in patients who have been diagnosed as having PTSD based upon the administration of any of the following tests: Clinician-Administered PTSD Scale Part 2 (CAPS) and the patient-rated Impact of Event Scale (IES). It is further contemplated that the compounds of the invention will be effective in inducing improvements in the scores of the CAPS, IES, CGI-Severity of Illness or CGI-Global Improvement tests. It is also contemplated that the compounds of this invention will be effective in preventing relapse of PTSD.

The compounds of the invention may be used to prevent, control or treat schizophrenia, paranoia or other related disorders of dopamine transmission.

The compounds can be administered in combination with anti-anxiety agents. Classes of anti-anxiety agents include: benzodiazepines (e.g. alprazolam (Xanax®), chlordiazepoxide (Librium®), clonazepam, chlorazepate, diazepam, halazepam, lorazepam, oxazepram, and prazepam, and pharmaceutically acceptable salts thereof);

5-HT1A agonist or antagonist, especially 5HT1A partial agonists (e.g. the 5-HT1A receptor partial agonists buspirone, flesinoxan, gepirone and ipsapirone, and pharmaceutically acceptable salts thereof);

corticotropin releasing factor (CRF) antagonists (including those described in WO 94/13643, WO 94/13644, WO 94/13661, WO 94/13676, and WO 94/13677);

phenothiazines (including promethazine, chlorpromazine, and trifluoperazine);

monoamine oxidase inhibitors (MAOIs, e.g. isocarboxazid (Marplan®), phenelzine (Nardil®), tranylcypromine (Parnate®) and selegiline, and pharmaceutically acceptable salts thereof);

reversible inhibitors of monoamine oxidase (RIMAs, e.g. moclobemide and pharmaceutically acceptable salts thereof);

tricyclic antidepressants (TCAs, e.g. amitriptyline (Elavil®), amoxapine, clomipramine, desipramine (Norpramin®), doxepin, imipramine (Tofranil®), maptroline, nortriptyline (Aventyl® and Pamelor®), perphenazine, protriptyline, and trimipramine (Surmentil®) and pharmaceutically acceptable salts thereof));

atypical antidepressants including bupropion, lithium, nefazodone, trazodone and viloxazine, and pharmaceutically acceptable salts thereof;

and selective serotonin reuptake inhibitors (SSRIs, e.g. paroxetine (Paxil®), venlafaxine, fluvoxamine, fluoxetine (Prozac®), citalopram (Celexa®), escitalopram, and sertraline (Zoloft®) and pharmaceutically acceptable salts thereof).

The compounds can also be used in a co-therapy with a second agent that has analgesic activity. Analgesics which can be used in co-therapy include, but are not limited to: NSAIDs (e.g., acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamnic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam, tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac), a non-narcotic analgesic such as tramadol, an opioid or narcotic analgesic (e.g., APF112, beta funaltrexamine, buprenorphine, butorphanol, codeine, cypridime, dezocine, dihydrocodeine, diphenyloxylate, enkephalin pentapeptide, fedotozine, fentanyl, hydrocodone, hydromorphone, levorphanol, loperamide, meperidine, mepivacaine, methadone, methyl nalozone, morphine, nalbuphine, nalmefene, naloxonazine, naloxone, naltrexone, naltrindole, nor-binaltorphimine, oxycodone, oxymorphone, pentazocine, propoxyphene, and trimebutine), NK1 receptor antagonists (e.g., ezlopitant and SR-14033, SSR-241585), CCK receptor antagonists (e.g., loxiglumide), NK3 receptor antagonists (e.g., talnetant, osanetant SR-142801, SSR-241585), norepinephrine-serotonin reuptake inhibitors (NSRI; e.g., milnacipran), vanilloid receptor agonists and antagonists, cannabinoid receptor agonists (e.g., arvanil), sialorphin, compounds or peptides that are inhibitors of neprilysin, frakefamide (H-Tyr-D-Ala-Phe(F)-Phe-NH₂; WO 01/019849 A1), Tyr-Arg (kyotorphin), CCK receptor agonists (e.g., caerulein), conotoxin peptides, peptide analogs of thymulin, dexloxiglumide (the R-isomer of loxiglumide; WO 88/05774), and analgesic peptides (e.g., endomorphin-1, endomorphin-2, nocistatin, dalargin, lupron, and substance P).

In addition, certain antidepressants can be used in co-therapy either because they have analgesic activity or are otherwise beneficial to use in combination with an analgesic. Examples of such anti-depressants include: selective serotonin reuptake inhibitors (e.g., fluoxetine, paroxetine, sertraline), serotonin-norepinephrine dual uptake inhibitors, venlafaxine and nefazadone. Certain anti-convulsants have analgesic activity and are useful in co-therapy. Such anti-convulsants include: gabapentin, carbamazepine, phenytoin, valproate, clonazepam, topiramate and lamotrigine. Such agents are considered particularly useful for treatment of neuropathic pain, e.g., treatment of trigeminal neuralgia, postherpetic neuralgia, and painful diabetic neuropathy. Additional compounds useful in co-therapy include: alpha-2-adrenergic receptor agonists (e.g., tizanidine and clonidine), mexiletine, corticosteroids, compounds that block the NMDA (N-methyl-Daspartate) receptor (e.g, dextromethorphan, ketamine, and amantadine), glycine antagonists, carisoprodol, cyclobenzaprine, various opiates, nonopioid antitussive (e.g. dextromethorphan, carmiphen, caramiphen and carbetapentane), opioid antitussives (e.g. codeine, hydrocodone, metaxolone. The compounds of the invention can also be combined with inhalable gaseous nitric oxide (for treating pulmonary vasoconstriction or airway constriction), a thromboxane A2 receptor antagonist, a stimulant (i.e. caffeine), an H₂—antagonist (e.g. ranitidine), an antacid (e.g. aluminum or magnesium hydroxide), an antiflatulent (e.g. simethicone), a decongestant (e.g. phenylephrine, phenylpropanolamine, pseudophedrine, oxymetazoline, ephinephrine, naphazoline, xylometazoline, propylhexedrine, or levodesoxyephedrine), a prostaglandin (e.g. misoprostol, enprostil, rioprostil, omoprostol or rosaprostol), a diuretic, a sedating or non-sedating histamine HI receptor antagonists/antihistamines (i.e. any compound that is capable of blocking, inhibiting, reducing or otherwise interrupting the interaction between histamine and its receptor) including but not limited to:—4 astemizole, acrivastine, antazoline, astemizole, azatadine, azelastine, astamizole, bromopheniramine, bromopheniramine maleate, carbinoxamine, carebastine, cetirizine, chlorpheniramine, chloropheniramine maleate, cimetidine, clemastine, cyclizine, cyproheptadine, descarboethoxyloratadine, dexchlorpheniramine, dimethindene, diphenhydramine, diphenylpyraline, doxylamine succinate, doxylamine, ebastine, efletirizine, epinastine, famotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norastemizole, noraztemizole, phenindamine, pheniramine, picumast, promethazine, pynlamine, pyrilamine, ranitidine, temelastine, terfenadine, trimeprazine, tripelenamine, and triprolidine; a 5HT1 agonist, such as a triptan (e.g. sumatriptan or naratriptan), an adenosine Al agonist, an EP ligand, a sodium channel blocker (e.g. lamotrigine), a substance P antagonist (e.g. an NK antagonist), a cannabinoid, a 5-lipoxygenase inhibitor, a leukotriene receptor antagonist/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (Singulair®), pranlukast, iralukast, pobilukast, SKB-106,203 and compounds described as having LTD4 antagonizing activity described in U.S. Pat. No. 5,565,473, a DMARD (e.g. methotrexate), a neurone stabilising antiepileptic drug, a mono-aminergic uptake inhibitor (e.g. venlafaxine), a matrix metalloproteinase inhibitor, a nitric oxide synthase (NOS) inhibitor, such as an iNOS or an nNOS inhibitor, an inhibitor of the release, or action, of tumor necrosis factor, an antibody therapy, such as a monoclonal antibody therapy, an antiviral agent, such as a nucleoside inhibitor (e.g. lamivudine) or an immune system modulator (e.g. interferon), a local anaesthetic, a known FAAH inhibitor (e.g., PMSF, URB532, URB597, or BMS-1, as well as those described in those described in WO04033652, U.S. Pat. No. 6,462,054, US20030092734, US20020188009, US20030195226, and WO04033422), an antidepressant (e.g., VPI-013), a fatty acid amide (e.g. anandamide, N-palmitoyl ethanolamine, N-oleoyl ethanolamide, 2-arachidonoylglycerol, or oleamide), arvanil, analogs of anadamide and arvanil as described in US 20040122089, and a proton pump inhibitor (e.g., omeprazole, esomeprazole, lansoprazole, pantorazole and rabeprazole).

The compound of the invention can also be used in a co-therapy with a second agent that is a cannabinoid receptor antagonist to prevent and/or treat obesity and other appetite related disorders.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by min, h, days, or weeks. Thus, the two or more agents can be administered within min of each other or within 1, 2, 3, 6, 9, 12, 15, 18, or 24 h of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include arecoline, arginine, barium, betaine, bismuth, chloroprocaine, choline, clemizole, deanol, imidazole, and morpholineethanol. In one embodiment are tris salts.

The agents of the invention can be administered orally, e.g., as a tablet or cachet containing a predetermined amount of the active ingredient, pellet, gel, paste, syrup, bolus, electuary, slurry, “flash dosage”, capsule, powder, granules, as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof,

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W. R. Grace Co., Baltimore, Md. USA), a coagulated aerosol of synthetic silica (Degussa Co., Plano, Tex. USA), a pyrogenic silicon dioxide (CAB-O-SIL, Cabot Co., Boston, Mass. USA), or mixtures thereof,

ANTI-CAKING AGENTS: calcium silicate, magnesium silicate, silicon dioxide, colloidal silicon dioxide, talc, or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, camauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. 4,767,628), poly(ε-caprolactone) and poly(alkylene oxide) (U.S. 20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, “flash dosage”, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299,WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

Compositions for oral administration can be in the form a of a “flash dosage”, i.e., a solid dosage form that is administered orally, which rapidly disperses in the mouth, and hence does not require great effort in swallowing and allows the compound to be rapidly ingested or absorbed through the oral mucosal membranes. In some embodiments, suitable rapidly dispersing dosage forms are also used in other applications, including the treatment of wounds and other bodily insults and diseased states in which release of the medicament by externally supplied moisture is not possible.

“Flash dose” forms are known in the art; see for example, effervescent dosage forms and quick release coatings of insoluble microparticles in U.S. Pat. Nos. 5,578,322 and 4,607,697; freeze dried foams and liquids in U.S. Pat. Nos. 4,642,903 and 5,631,023; melt spinning of dosage forms in U.S. Pat. Nos. 4,855,326; 5,380,326; and 5,518,730; solid, freeform fabrication in U.S. Pat. No. 6,471,992; saccharide-based carrier matrix and a liquid binder in U.S. Pat. Nos. 5,587,172; 5,616,344; 6,277,406; and 5,622,719; and other forms known to the art.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U. S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

The agents can be a free acid or base, or a pharmacologically acceptable salt thereof Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in U.S. 20030198619, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract. The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Combinations for Co-Morbid Conditions

It will be appreciated by one skilled in the art that a therapy administered in combination with the compounds of the present invention can be directed to the same or a different disorder target as that being targeted by the compounds of the present invention.

Administration of the compound of the invention may be first, followed by the other therapy; or administration of the other therapy may be first or they may be administered simultaneously either in two separate compositions or combined in a single composition. The other therapy is any known in the art to treat, prevent, or reduce the symptoms of the targeted disorder, e.g., a sleep disorder, or other disorders, e.g., other CNS disorders. In addition, some embodiments of the present invention have compounds administered in combination with other known therapies for the target disorder. Furthermore, the other therapy includes any agent of benefit to the patient when administered in combination with the disclosed compound.

For example, in some embodiments where the other therapy is a drug, it is administered as a separate formulation or in the same formulation as the compound of the invention. A compound of the invention is administered in combination therapy with any one or more of commercially-available, over-the-counter or prescription medications, including, but not limited to antimicrobial agents, fungistatic agents, germicidal agents, hormones, antipyretic agents, antidiabetic agents, bronchodilators, antidiarrheal agents, antiarrhythmic agents, coronary dilation agents, glycosides, spasmolytics, antihypertensive agents, antidepressants, antianxiety agents, other psychotherapeutic agents, corticosteroids, analgesics, contraceptives, nonsteroidal anti-inflammatory drugs, blood glucose lowering agents, cholesterol lowering agents, anticonvulsant agents, other antiepileptic agents, immunomodulators, anticholinergics, sympatholytics, sympathominietics, vasodilatory agents, anticoagulants, antiarrhythmics, prostaglandins having various pharmacologic activities, diuretics, sleep aids, antihistaininic agents, antineoplastic agents, oncolytic agents, antiandrogens, antimalarial agents, antileprosy agents, and various other types of drugs. See Goodman and Gilman's The Basis of Therapeutics (Eighth Edition, Pergamon Press, Inc., USA, 1990) and The Merck Index (Eleventh Edition, Merck & Co., Inc., USA, 1989).

### Combinations useful in Treatment of Diabetes

Suitable agents of use in combination with a compound of the present invention include anti-diabetic agents such as (1) PPARγ agonists such as glitazones (e.g., ciglitazone; darglitazone; englitazone; isaglitazone (MCC-555); pioglitazone; rosiglitazone; troglitazone; BRL49653; CLX-0921; 5-BTZD, and GW-0207, LG-100641, and LY-300512, and the like and compounds disclosed in PCT publication Nos. WO97/10813, WO97/27857,WO97/28115,WO97/28137,WO97/27847,WO03/000685,WO03/027112, WO03/035602, WO03/048130,WO03/055867, and the like; (2) biguanides such as buformin; metformin; and phenformin, and the like; (3) protein tyrosine phosphatase-1B (PTP-1B) inhibitors, such as ISIS 113715, and those disclosed in WO03/032916, WO03/032982, WO03/041729, WO03/055883; (4) sulfonylureas such as acetohexamide; carbutamide; chlorpropamide; diabinese; glibenclamide; glipizide; glyburide (glibenclamide); glimepiride; gliclazide; glipentide; gliquidone; glisolamide; tolazamide; and tolbutamide, and the like; (5) meglitinides such as repaglinide, and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and A1-3688, and the like; (8) insulin secreatagogues such as linogliride; and A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH2), and the like; (12) non-thiazolidinediones such as JT-501, and farglitazar (GW-2570/GI-262579), and the like; (13) PPARα/γ dual agonists such as BVT-142, CLX-0940, GW-1536, GW-1929, GW-2433, KRP-297, L-796449, LR-90, MK-0767, SB 219994, muraglitazar and reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976; and (14) other insulin sensitizing drugs; (15) VPAC2 receptor agonists; (16) GLK modulators, such as those disclosed in WO03/015774; (17) retinoid modulators such as those disclosed in WO03/000249; (18) GSK 3P/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP 1295884, EP 1295885, and the like; (19) glycogen phosphorylase (HGLPa) inhibitors, such as those disclosed in WO03/037864; (20) ATP consumption promotors such as those disclosed in WO03/007990; (21) TRB3 inhibitors, (22) vanilloid receptor ligands such as those disclosed in WO03/049702, (23) hypoglycemic agents such as those disclosed in WO03/015781, WO03/040114, (24) glycogen synthase kinase 3 inhibitors such as those disclosed in WO03/035663, (25) and agents such as those disclosed in WO99/51225 and US 20030134890; and WO01/24786, WO03/059870; (26) Insulin-responsive DNA binding protein-i (IRDBP-1) as disclosed in WO03/057827, and the like; (27) Adenosine A2 antagonists such as those disclosed in WO03/035639, WO03/035640, and the like.

### Combinations Useful in Treatment of Hyperlipidemia

Suitable agents of use in combination with a compound of the present invention include lipid lowering agents such as:

(1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, bervastatin, carvastatin, cerivastatin, crilvastatin, dalvastatin, fluvastatin, glenvastatin, itavastatin, lovastatin, mevastatin, pitavastatin, pravastatin, rivastatin, rosuvastatin, simvastatin, sirrivastatin, and ZD-4522, and the like and compounds disclosed in WO03/033481; (3) HMG-CoA synthase inhibitors; (4) cholesterol absorption inhibitors such as stanol esters, beta-sitosterol, sterol glycosides such as tiqueside; and azetidinones such as ezetimibe, and the like; (5) acyl coenzyme A-cholesterol acyl transferase (ACAT) inhibitors such as avasimibe (Current Opinion in Investigational Drugs. 3(9):291-297 (2003)), eflucimibe, KY505, SMP 797, CL-277,082 (Clin Pharmacol Ther. 48(2):189-94 (1990)) and the like; (6) CETP inhibitors such as JTT 705 identified as in Nature. 406, (6792):203-7 (2000), torcetrapib (CP-529,414 described in US20030186952 and WO2000017164), CP 532,632, BAY63-2149, SC 591, SC 795, and the like including those described in Current Opinion in Investigational Drugs. 4(3):291-297 (2003). (7) squalene synthetase inhibitors; (8) antioxidants such as probucol, AGI-1067 and the like; (9) PPARα agonists such as beclofibrate, benzafibrate, binifibrate, ciprofibrate, clinofibrate, clofibrate, etofibrate, fenofibrate, gemcabene, and gemfibrozil, lifibrol, GW 7647, BM 170744, LY518674; and other fibric acid derivatives, such as Atromid®, Lopid® and Tricor®, and those disclosed in WO03/033456, WO03/033481, WO03/043997, WO03/048116, WO03/053974, WO03/059864, WO03/05875, and the like; (10) FXR receptor modulators such as GW 4064, SR 103912, and the like; (11) LXR receptor modulators such as GW 3965, T9013137, and XTC0179628, and those disclosed in US20030125357, WO03/045382, WO03/053352, WO03/059874, and the like; (12) lipoprotein synthesis inhibitors such as niacin; (13) renin angiotensin system inhibitors; (14) PPARδ partial agonists, such as those disclosed in WO03/024395; (15) bile acid reabsorption inhibitors, such as BARI 1453, SC435, PHA384640, S8921, AZD7706, and the like; (16) PPARδ agonists such as GW 501516, and GW 590735, and the like, such as those disclosed in WO97/28149, WO01/79197, WO02/14291, WO02/46154, WO02/46176, WO02/076957, WO03/016291, WO03/033493; (17) triglyceride synthesis inhibitors; (18) microsomal triglyceride transport (MTTP) inhibitors, such as inplitapide, LAB687, and CP346086, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists; (25) PPAR modulators such as those disclosed in WO99/07357, WO99/11255, WO99/12534, WO99/15520, WO99/46232, WO00/12491, WO00/23442, WO0/236331, WO00/236332, WO00/218355, WO00/238553, WO01/25181, WO01/79150, WO02/79162, WO02/100403, WO02/102780, WO02/081428, WO03/016265, WO03/033453, WO03/042194, WO03/043997, WO03/066581, and the like; (26) niacin-bound chromium, as disclosed in WO03/039535; (27) substituted acid derivatives disclosed in WO03/040114; (28) apolipoprotein B inhibitors such as those disclosed in WO02/090347, WO02/28835, WO03/045921, WO03/047575; (29) Factor Xa modulators such as those disclosed in WO03/047517, WO03/047520, WO03/048081

### Combinations Useful in Treatment of Hypertension

Suitable agents of use in combination with a compound of the present invention include anti-hypertensive agents such as:

(1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as aprosartan, candesartan, eprosartan, irbesartan, losartan, olmesartan, pratosartan, tasosartan, telmisartan, valsartan, and EXP-3137, F16828K, and RNH6270, and the like; (9) α/β adrenergic blockers such as nipradilol, arotinolol and amosulalol, and the like; (10) alpha 1 blockers, such as terazosin, urapidil, prazosin, bunazosin, trimazosin, doxazosin, naftopidil, indoramin, WHP 164, and XENO010, and the like; (11) alpha 2 agonists such as lofexidine, tiamenidine, moxonidine, rilmenidine and guanobenz, and the like; (12) aldosterone inhibitors, and the like; and (13) angiopoietin-2-binding agents such as those disclosed in WO03/030833.

A number of embodiments of the invention have been described. Nevertheless, it will be understood that various modifications may be made without departing from the spirit and scope of the invention.

1. A method of inhibiting FAAH activity in a patient, the method comprising: administering celecoxib, valdecoxib, a compound structurally related to celecoxib or valdecoxib or a pharmaceutical composition comprising celecoxib, valdecoxib or a compound structurally related to celecoxib or valdecoxib to a patient at a dosage sufficient to inhibit FAAH activity in the patient.

2. The method of claim 1 wherein FAAH activity is inhibited at least 10%.

3. The method of claim 1 wherein FAAH activity is inhibited at least 20%.

4. The method of claim 1 wherein FAAH activity is inhibited at least 40%.

5. A method for treating anxiety in patient, the method comprising administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating anxiety.

6. A method for treating a sleep disorder in a patient, the method comprising administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating a sleep disorder.

7. The method of claim 6 wherein the sleep disorder is insomnia.

8. A method for treating intraocular hypertension in a patient, the method comprising administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating intraocular hypertension.

9. A method for treating anxiety in a patient, comprising:
(a) identifying a patient as suffering from anxiety or at risk of suffering from anxiety; and
(b) administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective to treat anxiety.

10. A method for treating a sleep disorder in a patient, comprising:
(a) identifying a patient as suffering from pain or at risk of suffering from a sleep disorder; and
(b) administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating a sleep disorder.

11. A method for treating a weight disorder in a patient, comprising:
(a) identifying a patient as suffering from a weight disorder; and
(b) administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating a weight disorder.

12. A method for treating intraocular hypertension in a patient, comprising:
(a) identifying a patient as suffering from intraocular hypotension or at risk of suffering from intraocular hypertension; and
(b) administering celecoxib, valdecoxib, a compound structurally related to celecoxib or valdecoxib or a pharmaceutical composition comprising celecoxib, valdecoxib or a compound structurally related to celecoxib or valdecoxib to the patient in an amount effective for treating intraocular hypertension.

13. The method of claim 5 wherein celecoxib, valdecoxib, a compound structurally related to celecoxib or valdecoxib or a pharmaceutical composition comprising celecoxib, valdecoxib or a compound structurally related to celecoxib or valdecoxib is administered at a dosage sufficient to inhibit FAAH activity.

14. The method of claim 1 wherein the compound has Formula I 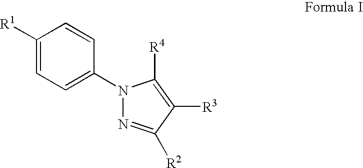
wherein: R¹ is sulfamyl; wherein R² is haloalkyl; wherein R³ is selected from hydrido, and alkyl; and wherein R⁴ is selected from aryl, cycloalkyl, and cycloalkenyl; wherein R⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy, hydroxyalkyl, haloalkoxy, sulfamyl, N-alkylsulfamyl, amino, N-alkylamino, N,N-dialkylamino, heterocyclic, nitro and acylamino; and pharmaceutically-acceptable salts thereof.

15. The method of claim 1 wherein the compound has Formula I or Formula II 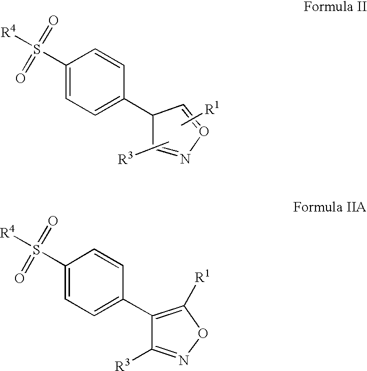
wherein: R¹ is selected from alkyl, carboxyalkyl, alkoxycarbonyl, aminocarbonyl, aminocarbonylalkyl, alkoxycarbonylalkyl, carboxyl, alkoxy, haloalkoxy, aralkoxy, cycloalkylalkoxy, alkylthio, aralkylthio, cycloalkylalkylthio, alkoxyalkyl, aralkoxyalkyl, alkylthioalkyl, aralkylthioalkyl, alkylaminoalkyl, aryloxyalkyl, arylthioalkyl, hydroxyl, amino, hydroxyalkyl, haloalkyl, cycloalkyl, cycloalkylalkyl, aralkyl, halo, alkylamino, aralkylamino, N-alkyl-N-aralkylamino, N-alkyl-N-cycloalkylalkylamino, arylcarbonyloxyalkyl, arylcarbonylthio, alkoxycarbonyloxyalkyl, alkylaminocarbonyloxyalkyl, alkoxycarbonylthioalkyl, and alkylaminocarbonylthioalkyl; R³ is selected from cycloalkyl, cycloalkenyl, and aryl, wherein R³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and R⁴ is selected from lower alkyl, hydroxyl, and amino; and pharmaceutically-acceptable salts thereof.
